(19) United States
(12) Patent Application Publication
(10) Pub. No.: US 2025/0059606 A1
(43) Pub. Date: Feb. 20, 2025

(54) ANALYSIS OF NUCLEIC ACID SEQUENCES

(71) Applicant: 10x Genomics, Inc., Pleasanton, CA (US)

(72) Inventors: Michael Schnall-Levin, San Francisco, CA (US); Mirna Jarosz, Palo Alto, CA (US); Christopher Hindson, Livermore, CA (US); Kevin Ness, Pleasanton, CA (US); Serge Saxonov, Oakland, CA (US); Benjamin Hindson, Pleasanton, CA (US); Xinying Zheng, Mountain View, CA (US); Patrick Marks, San Francisco, CA (US); John Stuelpnagel, Pleasanton, CA (US)

(21) Appl. No.: 18/934,042

(22) Filed: Oct. 31, 2024

Related U.S. Application Data
(63) Continuation of application No. 16/898,984, filed on Jun. 11, 2020, now Pat. No. 12,163,191.
Application No. 16/898,984 is a continuation of application No. 14/752,589, filed on Jun. 26, 2015, now abandoned.
(60) Provisional application No. 62/017,808, filed on Jun. 26, 2014.
(60) Provisional application No. 62/072,214, filed on Oct. 29, 2014.

(51) Int. Cl. C12Q 1/6883; C12Q 1/6827; G16B 30/00; G16B 30/10; G16B 30/20

(52) U.S. Cl. CPC C12Q 1/6883; C12Q 1/6827; G16B 30/00; G16B 30/10; G16B 30/20; C12Q 2535/122; C12Q 2600/156

(57) ABSTRACT

The present disclosure relates to methods, compositions and systems for haplotype phasing and copy number variation assays. Included within this disclosure are methods and systems for combining the barcode comprising beads with samples in multiple separate partitions, as well as methods of processing, sequencing and analyzing barcoded samples.

### CROSS-REFERENCE

This application is a continuation of U.S. patent application Ser. No. 16/898,984, filed Jun. 11, 2020, which is a continuation of U.S. patent application Ser. No. 14/752,589, filed Jun. 26, 2015, which claims priority to U.S. Provisional Patent Application No. 62/017,808, filed Jun. 26, 2014, and U.S. Provisional Patent Application No. 62/072,214, filed Oct. 29, 2014, each of which applications is herein incorporated by reference in its entirety for all purposes.

### BACKGROUND

A fundamental understanding of a particular human genome may require more than simply identifying the presence or absence of certain genetic variations such as mutations. It is also important to determine whether certain genetic variations appear on the same or different chromosomes (also known as phasing). Information about patterns of genetic variations, such as haplotypes is also important, as is information about the number of copies of genes.

The term “haplotype” refers to sets of DNA sequence variants (alleles) that are inherited together in contiguous blocks. In general, the human genome contains two copies of each gene-a maternal copy and a paternal copy. For a pair of genes each having two possible alleles, for example gene alleles “A” and “a”, and gene alleles “B” and “b”, the genome of a given individual will include one of two haplotypes, “AB/ab”, where the A and B alleles reside on the same chromosome (the “cis” configuration), or “Ab/aB”, where the A and B alleles reside on different chromosomes (the “trans” configuration). Phasing methods or assays can be used to determine whether a specified set of alleles reside on the same or different chromosomes. In some cases, several linked alleles that define a haplotype may correlate with, or be associated with, a particular disease phenotype; in such cases, a haplotype, rather than any one particular genetic variant, may be the most determinative factor as to whether a patient will display the disease.

Gene copy number also plays a role in some disease phenotypes. Most genes are normally present in two copies, however, amplified genes are genes that are present in more than two functional copies. In some instances, genes may also undergo a loss of functional copies. A loss or gain in gene copy number can lead to the production of abnormal levels of mRNA and protein expression, potentially leading to a cancerous state or other disorder. Cancer and other genetic disorders are often correlated with abnormal (increased or decreased) chromosome numbers (“aneuploidy”). Cytogenetic techniques such as fluorescence in situ hybridization or comparative genomic hybridization can be used to detect the presence of abnormal gene or chromosome copy numbers. Improved methods of detecting genetic phasing information, haplotypes or copy number variations are needed in the art.

### SUMMARY

The present disclosure provides methods and systems that may be useful in providing significant advances in the characterization of genetic material. These methods and systems can be useful in providing genetic characterizations that may be substantially difficult using generally available technologies, including, for example, haplotype phasing, identifying structural variations, e.g., deletions, duplications, copy-number variants, insertions, inversions, translocations, long tandem repeats (LTRs), short tandem repeats (STRs), and a variety of other useful characterizations.

An aspect of the disclosure provides a method for identifying one or more variations in a nucleic acid, comprising: a) providing a first fragment of the nucleic acid, wherein the first fragment has a length greater than 10 kilobases (kb); (b) sequencing a plurality of second fragments of the first fragment to provide a plurality of fragment sequences, which plurality of fragment sequences share a common barcode sequence; (c) attributing the plurality of fragment sequences to the first fragment by a presence of the common barcode sequence; (d) determining a nucleic acid sequence of the first fragment using the plurality of fragment sequences, wherein the nucleic acid sequence is determined at an error rate of less than 1%; and; (e) identifying the one or more variations in the nucleic acid sequence of the first fragment determined in (d), thereby identifying the one or more variations within the nucleic acid.

In some cases, the first fragment is in a discrete partition in among a plurality of discrete partitions. In some cases, the discrete partition is a droplet in an emulsion. In some cases the identifying comprises identifying phased variants in the nucleic acid sequence of the first fragment.

In some cases, the identifying comprises identifying one or more structural variations in the nucleic acid from the nucleic acid sequence of the first fragment. In some cases, the first fragment has a length greater than 15 kb. In some cases, the first fragment has a length greater than 20 kb. In some cases, the determining comprises mapping the plurality of fragment sequences to a reference. In some cases, the determining comprises assembling the plurality of fragment sequences with the common barcode sequence.

In some cases, the method for identifying one or more variations further comprises providing a plurality of first fragments of the nucleic acid that are at least 10 kb in length, and the identifying comprises determining a nucleic acid sequence from each of the plurality of first fragments and identifying the one or more variations in the nucleic acid from the nucleic acid sequence from each of the plurality of first fragments.

In some cases, the method for identifying one or more variations further comprises linking two or more nucleic acid sequences of the plurality of first fragments in an inferred contig based upon overlapping nucleic acid sequences of the two or more nucleic acid sequences, wherein the maximum inferred contig length is at least 10 kb. In some cases, the maximum inferred contig length is at least 20 kb. In some cases, the maximum inferred contig length is at least 40 kb. In some cases, the maximum inferred contig length is at least 50 kb. In some cases, the maximum inferred contig length is at least 100 kb. In some cases, the maximum inferred contig length is at least 200 kb. In some cases, the maximum inferred contig length is at least 500 kb. In some cases, the maximum inferred contig length is at least 750 kb. In some cases, the maximum inferred contig length is at least 1 megabase (Mb). In some cases, the maximum inferred contig length is at least 1.75 Mb. In some cases, the maximum inferred contig length is at least 2.5 Mb.

In some cases, the method for identifying one or more variations further comprises linking two or more nucleic acid sequences of the plurality of first fragments in a phase block based upon overlapping phased variants within the two or more nucleic acid sequences of the plurality of first fragments, wherein the maximum phase block length is at least 10 kb. In some cases, the maximum phase block length is at least 20 kb. In some cases, the maximum phase block length is at least 40 kb. In some cases, the maximum phase block length is at least 50 kb. In some cases, the maximum phase block length is at least 100 kb. In some cases, the maximum phase block length is at least 200 kb. In some cases, the maximum phase block length is at least 500 kb. In some cases, the maximum phase block length is at least 750 kb. In some cases, the maximum phase block length is at least 1 Mb. In some cases, the maximum phase block length is at least 1.75 Mb. In some cases, maximum phase block length is at least 2.5 Mb.

In some cases, the method for identifying one or more variations further comprises linking two or more nucleic acid sequences of the plurality of first fragments in an inferred contig based upon overlapping nucleic acid sequences of the two or more nucleic acid sequences, thereby creating a population of inferred contigs, wherein the N50 of the population of inferred contigs is at least 10 kb.

In some cases, the N50 of the population of inferred contigs is at least 20 kb. In some cases, the N50 of the population of inferred contigs is at least 40 kb. In some cases, the N50 of the population of inferred contigs is at least 50 kb. In some cases, the N50 of the population of inferred contigs is at least 100 kb.

In some cases, the N50 of the population of inferred contigs is at least 200 kb. In some cases, the N50 of the population of inferred contigs is at least 500 kb. In some cases, the N50 of the population of inferred contigs is at least 750 kb. In some cases, the N50 of the population of inferred contigs is at least 1 Mb. In some cases, the N50 of the population of inferred contigs is at least 1.75 Mb. In some cases, the N50 of the population of inferred contigs is at least 2.5 Mb.

In some cases, the method for identifying one or more variations further comprises linking two or more nucleic acid sequences of the plurality of first fragments in a phase block based upon overlapping phased variants within the two or more nucleic acid sequences of the plurality of first fragments, thereby creating a population of phase blocks, wherein the N50 of the population of phase blocks is at least 10 kb. In some cases, the N50 of the population of phase blocks is at least 20 kb. In some cases, the N50 of the population of phase blocks is at least 40 kb. In some cases, the N50 of the population of phase blocks is at least 50 kb. In some cases, the N50 of the population of phase blocks is at least 100 kb. In some cases, the N50 of the population of phase blocks is at least 200 kb. In some cases, the N50 of the population of phase blocks is at least 500 kb. In some cases, the N50 of the population of phase blocks is at least 750 kb. In some cases, the N50 of the population of phase blocks is at least 1 Mb. In some cases, the N50 of the population of phase blocks is at least 1.75 Mb. In some cases, the N50 of the population of phase blocks is at least 2.5 Mb.

An additional aspect of the disclosure provides a method of determining a presence of a structural variation of a nucleic acid. The method can comprise: (a) providing a plurality of first fragment molecules of the nucleic acid, wherein a given first fragment molecule of the plurality of first fragment molecules comprises the structural variation; (b) sequencing a plurality of second fragment molecules of each of the plurality of first fragment molecules to provide a plurality of fragment sequences, wherein each of the plurality of fragment sequences corresponding to a given first fragment molecule shares a common barcode sequence; and (c) determining the presence of the structural variation by (i) mapping the plurality of fragment sequences to a reference sequence, (ii) identifying the plurality of fragment sequences that share the common barcode sequence, and (iii) identifying the structural variation based on a presence of an elevated amount of the plurality of fragment sequences sharing the common barcode sequence that map to the reference sequence at locations that are further apart than a length of the given first fragment molecule, which elevated amount is relative to a sequence lacking the structural variation.

In some cases, the elevated amount is 1% or more with respect to a total number of the first fragment molecules that are derived from a region of the nucleic acid having the structural variation. In some cases, the elevated amount is 2% or more with respect to the total number of the first fragment molecules that are derived from a region of the nucleic acid having the structural variation. In some cases, the locations are at least about 100 bases apart. In some cases, the locations are at least about 500 bases apart. In some cases, the locations are at least about 1 kilobase (kb) apart. In some cases, the locations are at least about 10 kb apart.

In some cases, the method of determining a presence of a structural variation of a nucleic acid further comprises identifying the structural variation by creating an assembly of the given first fragment molecule from the plurality of fragment sequences, wherein the plurality of fragment sequences are selected as inputs for the assembly based upon a presence of the common barcode sequence. In some cases, the assembly is created by generating a consensus sequence from the plurality of fragment sequences. In some cases, the structural variation comprises a translocation.

An additional aspect of the disclosure provides a method of characterizing a variant nucleic acid sequence. In some cases, the method can comprise: (a) fragmenting a variant nucleic acid to provide a plurality of first fragments having a length greater than 10 kilobases (kb); (b) separating the plurality of first fragments into discrete partitions; (c) creating a plurality of second fragments from each first fragment within its respective partition, the plurality of second fragments having a barcode sequence attached thereto, which barcode sequence within a given partition is a common barcode sequence; (d) sequencing the plurality of second fragments and the barcode sequences attached thereto, to provide a plurality of second fragment sequences; (e) attributing the second fragment sequences to an original first fragment based at least in part on the presence of the common barcode sequence to provide a first fragment sequence context for the second fragment sequences; and (f) identifying a variant portion of the variant nucleic acid from the first fragment sequence context, thereby characterizing the variant nucleic acid sequence. In some cases, the attributing comprises assembling at least a portion of a sequence for an individual fragment from the plurality of first fragments from the plurality of second fragment sequences based, at least in part, on the presence of the common barcode sequence. In some cases, the attributing comprises mapping the plurality of second fragment sequences to an individual first fragment from the plurality of first fragments based at least in part upon the common barcode sequence.

In some cases, the method of characterizing a variant nucleic acid sequence further comprises linking two or more of the plurality of first fragments into an inferred contig, based upon overlapping sequence between the two or more of the plurality of first fragments. In some cases, the identifying comprises identifying one or more phased variants from the first fragment sequence context. In some cases, the method of characterizing a variant nucleic acid sequence further comprises linking two or more of the plurality of first fragments into a phase block, based upon overlapping phased variants between the two or more of the plurality of first fragments. In some cases, the identifying comprises identifying one or more structural variations from the first fragment sequence context. In some cases, the one or more structural variations are independently selected from insertions, deletions, translocations, retrotransposons, inversions, and duplications. In some cases, the structural variation comprises an insertion or a translocation, and the first fragment sequence context indicates a presence of the insertion or translocation.

An additional aspect of the disclosure provides a method of identifying variants in a sequence of a nucleic acid. In some cases, the method comprises: obtaining nucleic acid sequences of a plurality of individual fragment molecules of the nucleic acid, the nucleic acid sequences of the plurality of individual fragment molecules each having a length of at least 1 kilobase (kb); linking sequences of one or more of the plurality of individual fragment molecules in one or more inferred contigs; and identifying one or more variants from the one or more inferred contigs. In some cases, the obtaining comprises obtaining the nucleic acid sequences of a plurality of fragment molecules that are greater than 10 kb in length. In some cases, the obtaining comprises: providing a plurality of barcoded fragments of each individual fragment molecule of the plurality of individual fragment molecules, the barcoded fragments of a given individual fragment molecule having a common barcode; sequencing the plurality of barcoded fragments of the plurality of individual fragment molecules, the sequencing providing a sequencing error rate of less than 1%; and determining a sequence of the plurality of individual fragment molecules from sequences of the plurality of barcoded fragments and their associated barcodes.

In some cases, the linking comprises identifying one or more overlapping sequences between two or more individual fragment molecules to link the two or more individual fragment molecules into the one or more inferred contigs. In some cases, the linking comprises identifying one or more common variants between two or more individual fragment molecules to link the two or more individual fragment molecules into the one or more inferred contigs. In some cases, the one or more common variants are phased variants, and the one or more inferred contigs comprise a maximum phase block length of at least 100 kb. In some cases, the one or more variants identified in the identifying comprise structural variations. In some cases, the structural variations are selected from insertions, deletions, translocations, retrotransposons, inversions, and duplications.

An additional aspect of the disclosure provides a method of characterizing nucleic acids. In some cases, the method comprises: obtaining nucleic acid sequences of a plurality of fragment molecules having a length of at least 10 kilobases (kb); identifying one or more phased variant positions in the nucleic acid sequences of the plurality of fragment molecules; linking the nucleic acid sequences of at least a first fragment molecule to at least a second fragment molecule based upon a presence of one or more common phased variant positions within the first and second fragment molecules, to provide a phase block with a maximum phase block length of at least 10 kb; and identifying one or more phased variants from the phase block with the maximum phase block length of at least 10 kb. In some cases, the method of characterizing nucleic acids further comprises identifying one or more additional phased variants from the phase block. In some cases, the plurality of fragment molecules are in discrete partitions. In some cases, the discrete partitions are droplets in an emulsion. In some cases, the length of the plurality of fragment molecules is at least 50 kb. In some cases, the length of the plurality of fragment molecules is at least 100 kb. In some cases, the maximum phase block length is at least 50 kb. In some cases, the maximum phase block length is at least 100 kb. In some cases, the maximum phase block length is at least 1 Mb. In some cases, the maximum phase block length is at least 2 Mb. In some cases, the maximum phase block length is at least 2.5 Mb.

An additional aspect of the disclosure provides a method comprising: (a) partitioning a first nucleic acid into a first partition, where the first nucleic acid comprises the target sequence derived from a first chromosome of an organism; (b) partitioning a second nucleic acid into a second partition, where the second nucleic acid comprises the target sequence derived from a second chromosome of the organism; (c) in the first partition, attaching a first barcode sequence to fragments of the first nucleic acid or to copies of portions of the first nucleic acid to provide first barcoded fragments; (d) in the second partition, attaching a second barcode sequence to fragments of the second nucleic acid or to copies of portions of the second nucleic acid to provide second barcoded fragments, the second barcode sequence being different from the first barcode sequence; (e) determining the nucleic acid sequence of the first and second barcoded fragments, and assembling a nucleic acid sequence of the first and second nucleic acids; and (f) comparing the nucleic acid sequence of the first and second nucleic acids to characterize the first and second nucleic acids as deriving from first and second chromosomes, respectively. In some cases, oligonucleotides comprising the first barcode sequence are co-partitioned with the first nucleic acid, and oligonucleotides comprising the second barcode sequence are co-partitioned with the second nucleic acid. In some cases, the oligonucleotides comprising the first barcode sequence are releasably attached to a first bead, and the oligonucleotides comprising the second barcode sequence are releasably attached to a second bead, and the co-partitioning comprises co-partitioning the first and second beads into the first and second partitions, respectively. In some cases, the first and second partitions comprise droplets in an emulsion. In some cases, the first chromosome is a paternal chromosome and the second chromosome is a maternal chromosome. In some cases, the first chromosome and the second chromosome are homologous chromosomes. In some cases, the first nucleic acid and the second nucleic acid comprise one or more variations.

In some cases, the first and second chromosomes are derived from a fetus. In some cases, the first and second nucleic acids are obtained from a sample taken from a pregnant woman. In some cases, the first chromosome is chromosome 21, 18, or 13. In some cases, the second chromosome is chromosome 21, 18, or 13. In some cases, the method further comprises determining the relative quantity of the first or second chromosome. In some cases, the method further comprises determining the quantity of the first or second chromosome relative to a reference chromosome. In some cases, the first chromosome or second chromosome, or both, has an increase in copy number. In some cases, the increase in copy number is a result of cancer or aneuploidy. In some cases, the first chromosome or second chromosome, or both, has a decrease in copy number. In some cases, the decrease in copy number is a result of cancer or aneuploidy.

An additional aspect of the disclosure provides a method comprising: (a) partitioning a first nucleic acid into a first partition, where the first nucleic acid comprises the target sequence derived from a first chromosome of an organism; (b) partitioning a second nucleic acid into a second partition, where the second nucleic acid comprises the target sequence derived from a second chromosome of the organism; (c) in the first partition, attaching a first barcode sequence to fragments of the first nucleic acid or to copies of portions of the first nucleic acid to provide first barcoded fragments; (d) in the second partition, attaching a second barcode sequence to fragments of the second nucleic acid or to copies of portions of the second nucleic acid to provide second barcoded fragments, the second barcode sequence being different from the first barcode sequence; (e) determining the nucleic acid sequence of the first and second barcoded fragments, and assembling a nucleic acid sequence of the first and second nucleic acids; and (f) comparing the nucleic acid sequence of the first and second nucleic acids to identify any variation between the nucleic acid sequence of the first and second nucleic acids. In some cases, oligonucleotides comprising the first barcode sequence are co-partitioned with the first nucleic acid, and oligonucleotides comprising the second barcode sequence are co-partitioned with the second nucleic acid. In some cases, the oligonucleotides comprising the first barcode sequence are releasably attached to a first bead, and the oligonucleotides comprising the second barcode sequence are releasably attached to a second bead, and the co-partitioning comprises co-partitioning the first and second beads into the first and second partitions, respectively. In some cases, the first and second partitions comprise droplets in an emulsion. In some cases, the first chromosome is a paternal chromosome and the second chromosome is a maternal chromosome. In some cases, first chromosome and the second chromosome are homologous chromosomes. In some cases, the first nucleic acid and the second nucleic acid comprise one or more variations. In some cases, the first and second chromosomes are derived from a fetus. In some cases, the first and second nucleic acids are obtained from a sample taken from a pregnant woman. In some cases, the first chromosome is chromosome 21, 18, or 13. In some cases, the second chromosome is chromosome 21, 18, or 13. In some cases, the method further comprises determining the relative quantity of the first or second chromosome. In some cases, the method further comprises determining the quantity of the first or second chromosome relative to a reference chromosome. In some cases, the first chromosome or second chromosome, or both, has an increase in copy number. In some cases, the increase in copy number is a result of cancer or aneuploidy. In some cases, the first chromosome or second chromosome, or both, has a decrease in copy number. In some cases, the decrease in copy number is a result of cancer or aneuploidy.

An additional aspect of the disclosure provides a method for characterizing a fetal nucleic acid sequence. In some cases, the method comprises: (a) determining a maternal nucleic acid sequence, wherein the maternal nucleic acid is derived from a pregnant mother of a fetus, by: (i) fragmenting a maternal nucleic acid to provide a plurality of first maternal fragments; (ii) separating the plurality of first maternal fragments into maternal partitions; (iii) creating a plurality of second maternal fragments from each of the first maternal fragments within their respective maternal partitions, the plurality of second maternal fragments having a first barcode sequence attached thereto, wherein within a given maternal partition of the maternal partitions the second maternal fragments comprise a first common barcode sequence attached thereto; (iv) sequencing the plurality of second maternal fragments to provide a plurality of maternal fragment sequences; (v) attributing the maternal fragment sequences to an original first maternal fragment based at least in part on the presence of the first common barcode sequence to determine the maternal nucleic acid sequence; (b) determining a paternal nucleic acid sequence, wherein the paternal nucleic acid is derived from a father of the fetus, by: (i) fragmenting a paternal nucleic acid to provide a plurality of first paternal fragments; (ii) separating the plurality of first paternal fragments into paternal discrete partitions; (iii) creating a plurality of second paternal fragments from each first paternal fragment within its respective partition, the plurality of second paternal fragments having a second barcode sequence attached thereto, wherein within a given paternal partition, the second paternal fragments comprise a second common barcode sequence attached thereto; (iv) sequencing the plurality of second paternal fragments and the second barcode sequences attached thereto, to provide a plurality of paternal fragment sequences; (v) attributing the paternal fragment sequences to an original first paternal fragment based at least in part on the presence of the second common barcode sequence to determine the paternal nucleic acid sequence; (c) obtaining a fetal nucleic acid from the pregnant mother and determining a sequence of the fetal nucleic acid and/or one or more genetic variations of the sequence of the fetal nucleic acid using the maternal nucleic acid sequence and the paternal nucleic acid sequence.

In some cases, the paternal fragment sequences and the maternal fragment sequences are each used to link sequences into one or more inferred contigs. In some cases, the inferred contigs are used to construct maternal and paternal phase blocks. In some cases, the sequence of the fetal nucleic acid is compared to the maternal and paternal phase blocks to construct fetal phase blocks. In some cases, the paternal fragment sequences are assembled to produce at least a portion of sequences for the plurality of first paternal fragments, thereby determining the paternal nucleic acid sequence, and wherein the maternal fragment sequences are assembled to produce at least a portion of sequences for the plurality of first maternal fragments, thereby determining the maternal nucleic acid sequence. In some cases, the determining the paternal nucleic acid sequence comprises mapping the paternal fragment sequences to a paternal reference, and wherein the determining the maternal nucleic acid sequence comprises mapping the maternal fragment sequences to a maternal reference.

In some cases, the sequence of the fetal nucleic acid is determined with an accuracy of at least 99%. In some cases, the one or more genetic variations of the sequence of the fetal nucleic acid are determined with an accuracy of at least 99%. In some cases, the one or more genetic variations are selected from the group consisting of a structural variation and a single nucleotide polymorphism (SNP). In some cases, the one or more genetic variations are a structural variation selected from the group consisting of a copy number variation, an insertion, a deletion, a translocation, a retrotransposon, an inversion, a rearrangement, a repeat expansion and a duplication.

In some cases, the method for characterizing the fetal nucleic acid sequence further comprises, in (c), determining the one or more genetic variations of the sequence of the fetal nucleic acid using one or more genetic variations determined for the maternal nucleic acid sequence and the paternal nucleic acid sequence. In some cases, the method for characterizing the fetal nucleic acid sequence further comprises, in (c), determining one or more de novo mutations of the fetal nucleic acid. In some cases, the method for characterizing the fetal nucleic acid sequence further comprises, during or after (c), determining an aneuploidy associated with the fetal nucleic acid.

In some cases, the method for characterizing the fetal nucleic acid sequence further comprises, during or after (v) in (a), haplotyping the maternal nucleic acid sequence to provide a haplotype-resolved maternal nucleic acid sequence and, during or after (v) in (b), haplotyping the paternal nucleic acid sequence to provide a haplotype-resolved paternal nucleic acid sequence. In some cases, the method for characterizing the fetal nucleic acid sequence further comprises in (c), determining the sequence of the fetal nucleic acid and/or the one or more genetic variations using the haplotype-resolved maternal nucleic acid sequence and the haplotype-resolved paternal nucleic acid sequence. In some cases, one or more of the maternal nucleic acid and the paternal nucleic acid is genomic deoxyribonucleic acid (DNA). In some cases, in (c), the fetal nucleic acid comprises cell-free nucleic acid. In some cases, the method for characterizing the fetal nucleic acid sequence further comprises, in (a), determining the maternal nucleic acid sequence with an accuracy of at least 99%. In some cases, the method for characterizing the fetal nucleic acid sequence further comprises, in (b), determining the paternal nucleic acid sequence with an accuracy of at least 99%.

In some cases, the maternal nucleic acid sequence and/or the paternal nucleic acid sequence has a length greater than 10 kilobases (kb). In some cases, the maternal and paternal partitions comprise droplets in an emulsion. In some cases, in (a), the first barcode sequence is provided in the given maternal partition releasably attached to a first particle. In some cases, in (b), the second barcode sequence is provided in the given paternal partition releasably attached to a second particle.

An additional aspect of the disclosure provides a method for characterizing a sample nucleic acid. In some cases, the method comprises: (a) obtaining a biological sample from a subject, which biological sample includes a cell-free sample nucleic acid; (b) in a droplet, attaching a barcode sequence to fragments of the cell-free sample nucleic acid or to copies of portions of the sample nucleic acid, to provide barcoded sample fragments; (c) determining nucleic acid sequences of the barcoded sample fragments and providing a sample nucleic acid sequence based on the nucleic acid sequences of the barcoded sample fragments; (d) using a programmed computer processor to generate a comparison of the sample nucleic acid sequence to a reference nucleic acid sequence, which reference nucleic acid sequence has a length greater 10 kilobases (kb) and an accuracy of at least 99%; and (e) using the comparison to identify one or more genetic variations in the sample nucleic acid sequence, thereby associating the sample nucleic acid with a disease. In some cases, the one or more genetic variations in the sample nucleic acid sequence are selected from the group consisting of a structural variation and a single nucleotide polymorphism (SNP). In some cases, the one or more genetic variations of the sample nucleic acid sequence are a structural variation selected from the group consisting of a copy number variation, an insertion, a deletion, a retrotransposon, a translocation, an inversion, a rearrangement, a repeat expansion and a duplication. In some cases, in (c), the sample nucleic acid sequence is provided with an accuracy of at least 99%. In some cases, in (b), the barcode sequence is provided in the droplet releasably attached to a particle, and wherein (b) further comprises releasing the barcode sequence from the particle into the droplet prior to the attaching the barcode sequence. In some cases, in (b), the barcode sequence is provided as a portion of a primer sequence releasably attached to the particle, wherein the primer sequence also includes a random N-mer sequence, and wherein (b) further comprises releasing the primer sequence from the particle into the droplet prior to the attaching the barcode sequence. In some cases, in (b), attaching the barcode sequence to the fragments of the cell-free sample nucleic acid or to the copies of portions of the cell-free sample nucleic acid in an amplification reaction using the primer.

In some cases, the method for characterizing the sample nucleic acid further comprises: (i) in an additional droplet, attaching an additional barcode sequence to fragments of a reference nucleic acid or to copies of portions of the reference nucleic acid to provide barcoded reference fragments; and (ii) determining nucleic acid sequences of the barcoded reference fragments and determining the reference nucleic acid sequence based on the nucleic acid sequences of the barcoded reference fragments. In some cases, the determining the reference nucleic acid sequence comprises assembling the nucleic acid sequences of the barcoded reference fragments. In some cases, the method for characterizing the sample nucleic acid further comprises providing the additional barcode sequence in the additional droplet releasably attached to a particle and releasing the additional barcode sequence from the particle into the additional partition prior to the attaching the additional barcode sequence. In some cases, the method for characterizing the sample nucleic acid further comprises providing the additional barcode sequence as a portion of a primer sequence releasably attached to the particle, wherein the primer sequence also includes a random N-mer sequence, and releasing the primer from the particle into the additional droplet prior to the attaching the additional barcode sequence. In some cases, the method for characterizing the sample nucleic acid further comprises attaching the additional barcode sequence to the fragments of the reference nucleic acid or to the copies of portions of the reference nucleic acid in an amplification reaction using the primer. In some cases, the method for characterizing the sample nucleic acid further comprises determining one or more genetic variations in the reference nucleic acid sequence.

In some cases, the one or more genetic variations in the reference nucleic acid sequence are selected from the group consisting of a structural variation and a single nucleotide polymorphism (SNP). In some cases, the one or more genetic variations in the reference nucleic acid sequence are a structural variation selected from the group consisting of a copy number variation, an insertion, a deletion, a retrotransposon, a translocation, an inversion, a rearrangement, a repeat expansion and a duplication. In some cases, the reference nucleic acid comprises a germline nucleic acid sequence. In some cases, the reference nucleic acid comprises a cancer nucleic acid sequence. In some cases, the sample nucleic acid sequence has a length of greater than 10 kb. In some cases, the reference nucleic acid is derived from a genome indicative of an absence of a disease state. In some cases, the reference nucleic acid is a derived from a genome indicative of a disease state. In some cases, the disease state comprises cancer. In some cases, the disease state comprises an aneuploidy. In some cases, the cell-free sample nucleic acid comprises tumor nucleic acid. In some cases, the tumor nucleic acid comprises a circulating tumor nucleic acid.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 provides a schematic illustration of identification and analysis of phased variants using conventional processes versus example processes and systems described herein.

FIG. 2 provides a schematic illustration of the identification and analysis of structural variations using conventional processes versus example processes and systems described herein.

FIG. 3 illustrates an example workflow for performing an assay to detect copy number or haplotype using methods and compositions disclosed herein.

FIG. 4 provides a schematic illustration of an example process for combining a nucleic acid sample with beads and partitioning the nucleic acids and beads into discrete droplets

FIG. 5 provides a schematic illustration of an example process for barcoding and amplification of chromosomal nucleic acid fragments.

FIG. 6 provides a schematic illustration of an example use of barcoding of chromosomal nucleic acid fragments in attributing sequence data to individual chromosomes.

FIG. 7 provides a schematic illustration of an example of phased sequencing processes.

FIG. 8 provides a schematic illustration of an example subset of the genome of a healthy patient (top panel) and a cancer patient with a gain in haplotype copy number (central panel) or loss of haplotype copy number (bottom panel).

FIGS. 9A-B provides: (a) a schematic illustration showing a relative contribution of tumor DNA and (b) a representation of detecting such copy gains and losses by ordinary sequencing methods.

FIG. 10 provides a schematic illustration of an example of detecting copy gains and losses using a single variant position (left panel) and combined variant positions (right panel).

FIG. 11 provides a schematic illustration of the potential of described methods and systems to identify gains and losses in copy number.

FIG. 12 illustrates an example workflow for performing an aneuploidy test based on determination of chromosome number and copy number variation using methods and compositions described herein.

FIGS. 13A-B illustrate an example overview of a process for identifying structural variations such as translocations and gene fusions in genetic samples.

FIG. 14 illustrates an example workflow for performing a cancer diagnostic test based on determination of copy number variation using the methods and compositions described herein.

FIG. 15 provides a schematic illustration of an EML-4-ALK structural variation from an NCI-H2228 cancer cell line.

FIGS. 16A and 16B, provide barcode mapping data using the systems described herein for identifying the presence of the EML-4-ALK variant structure shown in FIG. 15, in the cancer cell line (FIG. 16A), as compared to a negative control cell line (FIG. 16B).

FIG. 17 schematically depicts an example workflow of analyzing a paternal nucleic acid sequence as described herein.

FIG. 18 schematically depicts an example workflow of analyzing a maternal nucleic acid sequence as described herein.

FIG. 19 schematically depicts an example workflow of analyzing a fetal nucleic acid sequence as described herein.

FIG. 20 schematically depicts an example workflow of analyzing a reference nucleic acid sequence as described herein.

FIG. 21 schematically depicts an example workflow of analyzing a sample nucleic acid sequence as described herein.

FIG. 22 schematically depicts an example computer control system.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used herein, the term “organism” generally refers to a contiguous living system. Non-limiting examples of organisms includes animals (e.g., humans, other types of mammals, birds, reptiles, insects, other example types of animals described elsewhere herein), plants, fungi and bacterium.

As used herein, the term “contig” generally refers to a contiguous nucleic acid sequence of a given length. The contiguous sequence may be derived from an individual sequence read, including either a short or long read sequence read, or from an assembly of sequence reads that are aligned and assembled based upon overlapping sequences within the reads, or that are defined as linked within a fragment based upon other known linkage data, e.g., the tagging with common barcodes as described elsewhere herein. These overlapping sequence reads may likewise include short reads, e.g., less than 500 bases, e.g., in some cases from approximately 100 to 500 bases, and in some cases from 100 to 250 bases, or based upon longer sequence reads, e.g., greater than 500 bases, 1000 bases or even greater than 10,000 bases.

### I. Overview

This disclosure provides methods and systems useful in providing significant advances in the characterization of genetic material. In some cases, the methods and systems can be useful in providing genetic characterizations that are very difficult or even impossible using generally available technologies, including, for example, haplotype phasing, identifying structural variations, e.g., deletions, duplications, copy-number variants, insertions, inversions, retrotransposons, translocations, LTRs, STRs, and a variety of other useful characterizations.

In general, the methods and systems described herein accomplish the above goals by providing for the sequencing of long individual nucleic acid molecules, which permit the identification and use of long range variant information, e.g., relating variations to different sequence segments, including sequence segments containing other variations, that are separated by significant distances in the originating sequence, e.g., longer than is provided by short read sequencing technologies. However, these methods and systems achieve these objectives with the advantage of extremely low sequencing error rates of short read sequencing technologies, and far below those of the reported long read-length sequencing technologies, e.g., single molecule sequencing, such as SMRT Sequencing and nanopore sequencing technologies.

In general, the methods and systems described herein segment long nucleic acid molecules into smaller fragments that are sequenceable using high-throughput, higher accuracy short-read sequencing technologies, but do such segmentation in a manner that allows the sequence information derived from the smaller fragments to be attributed to the originating longer individual nucleic acid molecules. By attributing sequence reads to an originating longer nucleic acid molecule, one can gain significant characterization information for that longer nucleic acid sequence, that one cannot generally obtain from short sequence reads alone. As noted, such characterization information can include haplotype phasing, identification of structural variations, and identifying copy number variations.

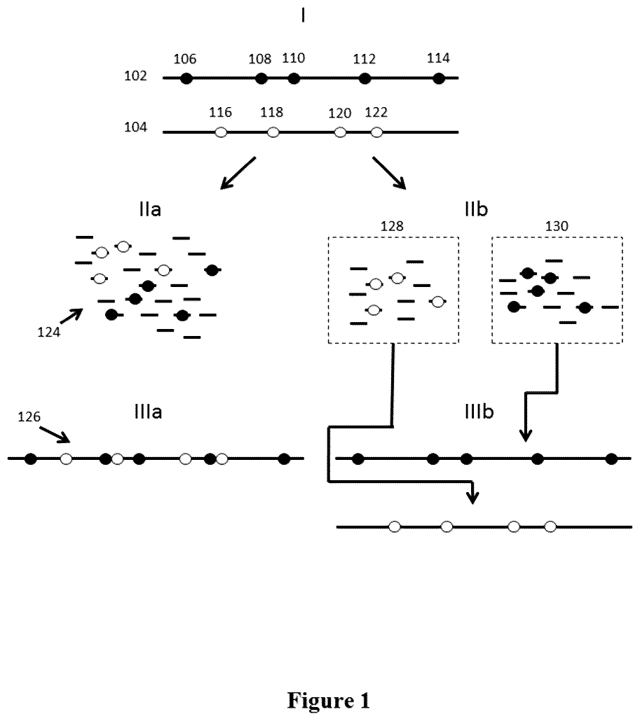

The advantages of the methods and systems described herein are described with respect to a number of general examples. In a first example, phased sequence variants are identified and characterized using the methods and systems described herein. FIG. 1 schematically illustrates the challenges of phased variant calling and the solutions presented by the methods described herein. As shown, nucleic acids 102 and 104 in Panel I represent two haploid sequences of the same region of different chromosomes, e.g., maternally and paternally inherited chromosomes. Each sequence includes a series of variants, e.g., variants 106-114 on nucleic acid 102, and variants 116-122 on nucleic acid 104, at different alleles that characterize each haploid sequence. Because of their very short sequence reads, most sequencing technologies are unable to provide the context of individual variants relative to other variants on the same haploid sequence. Additionally, because they rely on sample preparation techniques that do not separate individual molecular components, e.g., each haploid sequence, one is unable to identify the phasing of the various variants, e.g., the haploid sequence from which a variant derives. As a result, these short read technologies are unable to resolve these variants to their originating molecules. The difficulties with this approach are schematically illustrated in Panels IIa and IIIa. Briefly, pooled fragments from both haploid sequences, shown in Panel IIa, are sequenced, resulting in a large number of short sequence reads 124, and the resulting sequence 126 is assembled (shown in Panel IIIa). As shown, because one does not have the relative phasing context of any of the shorter sequence reads in Panel IIa, one would be unable to resolve the variants as between two different haploid sequences in the assembly process. Accordingly, the resulting assembly shown in Panel IIIa, results in single consensus sequence assembly 126, including all of variants 106-122.

In contrast, and as shown in Panel IIb of FIG. 1, the methods and systems described herein breakdown or segment the longer nucleic acids 102 and 104 into shorter, sequenceable fragments, as with the above described approach, but retain with those fragments the ability to attribute them to their originating molecular context. This is schematically illustrated in Panel IIb, in which different fragments are grouped or “compartmentalized” according to their originating molecular context. In the context of the disclosure, this grouping can be accomplished through one or both of physically partitioning the fragments into groups that retain the molecular context, as well as tagging those fragments in order to subsequently be able to elucidate that context.

This grouping is schematically illustrated as the allocation of the shorter sequence reads as between groups 128 and 130, representing short sequence reads from nucleic acids 102 and 104, respectively. Because the originating sequence context is retained through the sequencing process, one can employ that context in resolving the original molecular context, e.g., the phasing, of the various variants 106-114 and 116-122 as between sequences 102 and 104, respectively.

In another exemplary advantaged application, the methods and systems are useful in characterizing structural variants that are generally unidentifiable or at least difficult to identify, using short read sequence technologies.

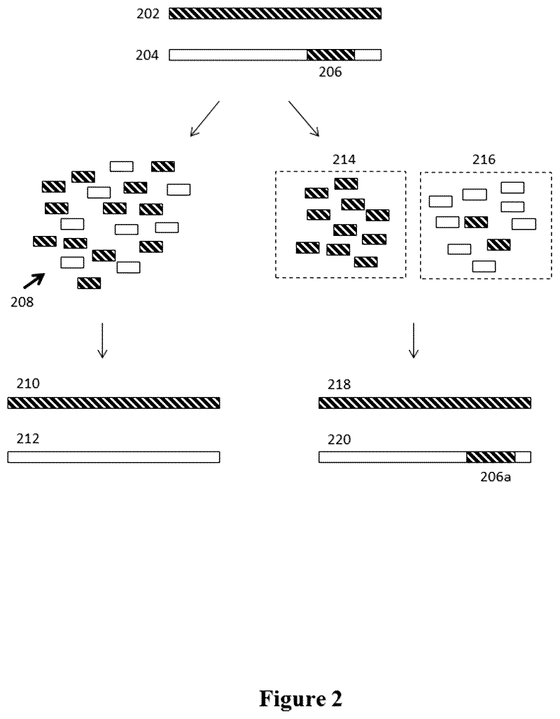

This is schematically illustrated with reference to a simple translocation event in FIG. 2. As shown, a genomic sample may include nucleic acids that include a translocation event, e.g., a translocation of genetic element 206 from sequence 202 to sequence 204. Such translocations may be any of a variety of different translocation types, including, for example, translocations between different chromosomes, whether to the same or different regions, between different regions of the same chromosome.

Again, as with the example illustrated in FIG. 1, above, conventional sequencing starts by breaking up the sequences 202 and 204 in Panel I into small fragments and producing short sequence reads 208 from those fragments, as shown in Panel IIa. Because these sequence fragments 208 are relatively short, the context of the translocated sequence 206, i.e., as originating from a variant location on the same or a different sequence, is easily lost during the assembly process. Further, because of their short read lengths, sequence assemblies are often predicated on the use of a reference sequence that would, almost by definition, not reflect structural variations. As such, the short sequence reads 208 would invariably be assembled to disregard the proper location of the translocated sequence 206, and would instead assemble the non-variant sequences 210 and 212, as shown in Panel IIIa.

In contrast, using the methods and systems described herein, the short sequence reads derived from sequences 202 and 204, are provided with a compartmentalization, shown in Panel IIb as groups 214 and 216, that retain the original molecular grouping of the smaller sequence fragments, allowing their assembly as sequences 218 and 220, shown in Panel IIIb, allowing attribution back to the originating sequences 202 and 204, and identification of the translocation variation, e.g., translocated sequence segment 206a in correct sequence assemblies 218 and 220, as illustrated in Panel IIIb.

As noted above, the methods and systems described herein provide individual molecular context for short sequence reads of longer nucleic acids. As used herein, individual molecular context refers to sequence context beyond the specific sequence read, e.g., relation to adjacent or proximal sequences, that are not included within the sequence read itself, and as such, will generally be such that they would not be included in whole or in part in a short sequence read, e.g., a read of about 150 bases, or about 300 bases for paired reads. In some aspects, the methods and systems provide long range sequence context for short sequence reads. Such long range context includes relationship or linkage of a given sequence read to sequence reads that are within a distance of each other of longer than 1 kilobase (kb), longer than 5 kb, longer than 10 kb, longer than 15 kb, longer than 20 kb, longer than 30 kb, longer than 40 kb, longer than 50 kb, longer than 60 kb, longer than 70 kb, longer than 80 kb, longer than 90 kb or even longer than 100 kb, or longer. By providing longer range individual molecular context, the methods and systems described herein also provide much longer inferred molecular context. Sequence context, as described herein can include lower resolution context, e.g., from mapping the short sequence reads to the individual longer molecules or contigs of linked molecules, as well as the higher resolution sequence context, e.g., from long range sequencing of large portions of the longer individual molecules, e.g., having contiguous determined sequences of individual molecules where such determined sequences are longer than 1 kb, longer than 5 kb, longer than 10 kb, longer than 15 kb, longer than 20 kb, longer than 30 kb, longer than 40 kb, longer than 50 kb, longer than 60 kb, longer than 70 kb, longer than 80 kb, longer than 90 kb or even longer than 100 kb. As with sequence context, the attribution of short sequences to longer nucleic acids, e.g., both individual long nucleic acid molecules or collections of linked nucleic acid molecules or contigs, may include both mapping of short sequences against longer nucleic acid stretches to provide high level sequence context, as well as providing assembled sequences from the short sequences through these longer nucleic acids.

Furthermore, while one may utilize the long range sequence context associated with long individual molecules, having such long range sequence context also allows one to infer even longer range sequence context. By way of one example, by providing the long range molecular context described above, one can identify overlapping variant portions, e.g., phased variants, translocated sequences, etc., among long sequences from different originating molecules, allowing the inferred linkage between those molecules. Such inferred linkages or molecular contexts are referred to herein as “inferred contigs”. In some cases when discussed in the context of phased sequences, the inferred contigs may represent commonly phased sequences, e.g., where by virtue of overlapping phased variants, one can infer a phased contig of substantially greater length than the individual originating molecules. These phased contigs are referred to herein as “phase blocks”.

By starting with longer single molecule reads, one can derive longer inferred contigs or phase blocks than would otherwise be attainable using short read sequencing technologies or other approaches to phased sequencing. See, e.g., published U.S. Patent Publication No. 2013/0157870, the full disclosure of which is herein incorporated by reference in its entirety. In particular, using the methods and systems described herein, one can obtain inferred contig or phase block lengths having an N50 (the contig or phase block length for which the collection of all phase blocks or contigs of that length or longer contain at least half of the sum of the lengths of all contigs or phase blocks, and for which the collection of all contigs or phase blocks of that length or shorter also contains at least half the sum of the lengths of all contigs or phase blocks), mode, mean, or median of at least about 10 kilobases (kb), at least about 20 kb, at least about 50 kb. In some aspects, inferred contig or phase block lengths have an N50, mode, mean, or median of at least about 100 kb, at least about 150 kb, at least about 200 kb, and in some cases, at least about 250 kb, at least about 300 kb, at least about 350 kb, at least about 400 kb, and in some cases, at least about 500 kb, at least about 750 kb, at least about 1 Mb, at least about 1.75 Mb, at least about 2.5 Mb or more, are attained. In still other cases, maximum inferred contig or phase block lengths of at least or in excess of 20 kb, 40 kb, 50 kb, 100 kb, 200 kb, 300 kb, 400 kb, 500 kb, 750 kb, 1 megabase (Mb), 1.75 Mb, 2 Mb or 2.5 Mb may be obtained. In still other cases, inferred contigs or phase blocks lengths can be at least about 20 kb, at least about 40 kb, at least about 50 kb, at least about 100 kb, at least about 200 kb, and in some cases, at least about 500 kb, at least about 750 kb, at least about 1 Mb, and in some cases at least about 1.75 Mb, at least about 2.5 Mb or more.

In one aspect, the methods and systems described herein provide for the compartmentalization, depositing or partitioning of sample nucleic acids, or fragments thereof, into discrete compartments or partitions (referred to interchangeably herein as partitions), where each partition maintains separation of its own contents from the contents of other partitions. Unique identifiers, e.g., barcodes, may be previously, subsequently or concurrently delivered to the partitions that hold the compartmentalized or partitioned sample nucleic acids, in order to allow for the later attribution of the characteristics, e.g., nucleic acid sequence information, to the sample nucleic acids included within a particular compartment, and particularly to relatively long stretches of contiguous sample nucleic acids that may be originally deposited into the partitions.

The sample nucleic acids can be partitioned such that the nucleic acids are present in the partitions in relatively long fragments or stretches of contiguous nucleic acid molecules. These fragments can represent a number of overlapping fragments of the overall sample nucleic acids to be analyzed, e.g., an entire chromosome, exome, or other large genomic fragment. These sample nucleic acids may include whole genomes, individual chromosomes, exomes, amplicons, or any of a variety of different nucleic acids of interest. In some cases, these fragments of the sample nucleic acids may be longer than 100 bases, longer 500 bases, longer than 1 kb, longer than 5 kb, longer than 10 kb, longer than 15 kb, longer than 20 kb, longer than 30 kb, longer than 40 kb, longer than 50 kb, longer than 60 kb, longer than 70 kb, longer than 80 kb, longer than 90 kb or even longer than 100 kb, which permits the longer range molecular context described above.

The sample nucleic acids can also be partitioned at a level whereby a given partition has a very low probability of including two overlapping fragments of the starting sample nucleic acid. This can be accomplished by providing the sample nucleic acid at a low input amount and/or concentration during the partitioning process. As a result, in some cases, a given partition may include a number of long, but non-overlapping fragments of the starting sample nucleic acids. The sample nucleic acids in the different partitions are then associated with unique identifiers, where for any given partition, nucleic acids contained therein possess the same unique identifier, but where different partitions may include different unique identifiers. Moreover, because the partitioning allocates the sample components into very small volume partitions or droplets, it will be appreciated that in order to achieve the allocation as set forth above, one need not conduct substantial dilution of the sample, as would can be required in higher volume processes, e.g., in tubes, or wells of a multiwell plate. Further, because the systems described herein employ such high levels of barcode diversity, one can allocate diverse barcodes among higher numbers of genomic equivalents, as provided above. In particular, previously described, multiwell plate approaches (see, e.g., U.S. Patent Publication No. 2013/0079231 and 2013/0157870, the full disclosures of which are herein incorporated by reference in their entireties) may only operate with a hundred to a few hundred different barcode sequences, and employ a limiting dilution process of their sample in order to be able to attribute barcodes to different cells/nucleic acids. As such, they generally operate with far fewer than 100 cells, which would can provide a ratio of genomes:(barcode type) on the order of 1:10, and certainly well above 1:100. The systems described herein, on the other hand, because of the high level of barcode diversity, e.g., in excess of 10,000, 100,000, 500,000, etc. diverse barcode types, can operate at genome:(barcode type) ratios that are on the order of 1:50 or less, 1:100 or less, 1:1000 or less, or even smaller ratios, while also allowing for loading higher numbers of genomes (e.g., on the order of greater than 100 genomes per assay, greater than 500 genomes per assay, 1000 genomes per assay, or even more) while still providing for far improved barcode diversity per genome.

Often, the sample is combined with a set of oligonucleotide tags that are releasably-attached to beads prior to the partitioning. The oligonucleotides may comprise at least a first and second region. The first region may be a barcode region that, as between oligonucleotides within a given partition, may be substantially the same barcode sequence, but as between different partitions, may and, in most cases is a different barcode sequence. The second region may be a an N-mer (e.g., either a random N-mer or an N-mer designed to target a particular sequence) that can be used to prime the nucleic acids within the sample within the partitions. In some cases, where the N-mer is designed to target a particular sequence, it may be designed to target a particular chromosome (e.g., chromosome 1, 13, 18, or 21), or region of a chromosome, e.g., an exome or other targeted region. In some cases, the N-mer may be designed to target a particular gene or genetic region, such as a gene or region associated with a disease or disorder (e.g., cancer). Within the partitions, an amplification reaction may be conducted using the second N-mer to prime the nucleic acid sample at different places along the length of the nucleic acid. As a result of the amplification, each partition may contain amplified products of the nucleic acid that are attached to an identical or near-identical barcode, and that may represent overlapping, smaller fragments of the nucleic acids in each partition. The bar-code can serve as a marker that signifies that a set of nucleic acids originated from the same partition, and thus potentially also originated from the same strand of nucleic acid. Following amplification, the nucleic acids may be pooled, sequenced, and aligned using a sequencing algorithm. Because shorter sequence reads may, by virtue of their associated barcode sequences, be aligned and attributed to a single, long fragment of the sample nucleic acid, all of the identified variants on that sequence can be attributed to a single originating fragment and single originating chromosome. Further, by aligning multiple co-located variants across multiple long fragments, one can further characterize that chromosomal contribution. Accordingly, conclusions regarding the phasing of particular genetic variants may then be drawn. Such information may be useful for identifying haplotypes, which are generally a specified set of genetic variants that reside on the same nucleic acid strand or on different nucleic acid strands. Copy number variations may also be identified in this manner.

The described methods and systems provide significant advantages over current nucleic acid sequencing technologies and their associated sample preparation methods. Haplotype phasing and copy number variation data are generally not available by sequencing genomic DNA because biological samples (blood, cells, or tissue samples, for example) are processed en masse to extract the genetic material from an ensemble of cells, and convert it into sequencing libraries that are configured specifically for a given sequencing technology. As a result of this ensemble sample processing approach, sequencing data generally provides non-phased genotypes, in which it is not possible to determine whether genetic information is present on the same or different chromosomes.

In addition to the inability to attribute genetic characteristics to a particular chromosome, such ensemble sample preparation and sequencing methods are also predisposed towards primarily identifying and characterizing the majority constituents in the sample, and are not designed to identify and characterize minority constituents, e.g., genetic material contributed by one chromosome, or by one or a few cells, or fragmented tumor cell DNA molecule circulating in the bloodstream, that constitute a small percentage of the total DNA in the extracted sample. The described methods and systems also provide a significant advantage for detecting minor populations that are present in a larger sample. As such, they can be useful for assessing copy number variations in a sample since often only a small portion of a clinical sample contains tissue with copy number variations. For example, if the sample is a blood sample from a pregnant woman, only a small fraction of the sample would contain circulating cell-free fetal DNA.

The use of the barcoding technique disclosed herein confers the unique capability of providing individual molecular context for a given set of genetic markers, i.e., attributing a given set of genetic markers (as opposed to a single marker) to individual sample nucleic acid molecules, and through variant coordinated assembly, to provide a broader or even longer range inferred individual molecular context, among multiple sample nucleic acid molecules, and/or to a specific chromosome. These genetic markers may include specific genetic loci, e.g., variants, such as SNPs, or they may include short sequences. Furthermore, the use of barcoding confers the additional advantages of facilitating the ability to discriminate between minority constituents and majority constituents of the total nucleic acid population extracted from the sample, e.g. for detection and characterization of circulating tumor DNA in the bloodstream, and also reduces or eliminates amplification bias during any amplification. In addition, implementation in a microfluidics format confers the ability to work with extremely small sample volumes and low input quantities of DNA, as well as the ability to rapidly process large numbers of sample partitions (e.g., droplets) to facilitate genome-wide tagging.

As described previously, an advantage of the methods and systems described herein is that they can achieve results through the use of ubiquitously available, short read sequencing technologies. Such technologies have the advantages of being readily available and widely dispersed within the research community, with protocols and reagent systems that are well characterized and highly effective. These short read sequencing technologies include those available from, e.g., Illumina, Inc. (e.g., GXII, NextSeq, MiSeq, HiSeq, X10), Ion Torrent division of Thermo-Fisher (e.g., Ion Proton and Ion PGM), pyrosequencing methods, as well as others.

Of particular advantage is that the methods and systems described herein utilize these short read sequencing technologies and do so with their associated low error rates. In particular, the methods and systems described herein achieve individual molecular read lengths or context, as described above, but with individual sequencing reads, excluding mate pair extensions, that are shorter than 1000 bp, shorter than 500 bp, shorter than 300 bp, shorter than 200 bp, shorter than 150 bp or even shorter; and with sequencing error rates for such individual molecular read lengths that are less than 5%, less than 1%, less than 0.5%, less than 0.1%, less than 0.05%, less than 0.01%, less than 0.005%, or even less than 0.001%.

### II. Work Flow Overview

In one exemplary aspect, the methods and systems described in the disclosure provide for depositing or partitioning individual samples (e.g., nucleic acids) into discrete partitions, where each partition maintains separation of its own contents from the contents in other partitions. As used herein, the partitions refer to containers or vessels that may include a variety of different forms, e.g., wells, tubes, micro or nanowells, through holes, or the like. In some aspects, however, the partitions are flowable within fluid streams. These vessels may be comprised of, e.g., microcapsules or micro-vesicles that have an outer barrier surrounding an inner fluid center or core, or they may be a porous matrix that is capable of entraining and/or retaining materials within its matrix. In some aspects, however, these partitions may comprise droplets of aqueous fluid within a non-aqueous continuous phase, e.g., an oil phase. A variety of different vessels are described in, for example, U.S. patent application Ser. No. 13/966,150, filed Aug. 13, 2013. Likewise, emulsion systems for creating stable droplets in non-aqueous or oil continuous phases are described in detail in, e.g., U.S. Patent Publication No. 2010/0105112, the full disclosure of which is herein incorporated by reference in its entirety. In certain cases, microfluidic channel networks can be suited for generating partitions as described herein. Examples of such microfluidic devices include those described in detail in U.S. Provisional Patent Application No. 61/977,804, filed Apr. 10, 2014, the full disclosure of which is incorporated herein by reference in its entirety for all purposes. Alternative mechanisms may also be employed in the partitioning of individual cells, including porous membranes through which aqueous mixtures of cells are extruded into non-aqueous fluids. Such systems are generally available from, e.g., Nanomi, Inc.

In the case of droplets in an emulsion, partitioning of sample materials, e.g., nucleic acids, into discrete partitions may generally be accomplished by flowing an aqueous, sample containing stream, into a junction into which is also flowing a non-aqueous stream of partitioning fluid, e.g., a fluorinated oil, such that aqueous droplets are created within the flowing stream partitioning fluid, where such droplets include the sample materials. As described below, the partitions, e.g., droplets, can also include co-partitioned barcode oligonucleotides. The relative amount of sample materials within any particular partition may be adjusted by controlling a variety of different parameters of the system, including, for example, the concentration of sample in the aqueous stream, the flow rate of the aqueous stream and/or the non-aqueous stream, and the like. The partitions described herein are often characterized by having extremely small volumes. For example, in the case of droplet based partitions, the droplets may have overall volumes that are less than 1000 picoliters (pL), less than 900 pL, less than 800 pL, less than 700 pL, less than 600 pL, less than 500 pL, less than 400pL, less than 300 pL, less than 200 pL, less than 100pL, less than 50 pL, less than 20 pL, less than 10 pL, or even less than 1 pL. Where co-partitioned with beads, it will be appreciated that the sample fluid volume within the partitions may be less than 90% of the above described volumes, less than 80%, less than 70%, less than 60%, less than 50%, less than 40%, less than 30%, less than 20%, or even less than 10% the above described volumes. In some cases, the use of low reaction volume partitions can be advantageous in performing reactions with very small amounts of starting reagents, e.g., input nucleic acids. Methods and systems for analyzing samples with low input nucleic acids are presented in U.S. Provisional Patent Application No. 62/017,580, filed Jun. 26, 2014, the full disclosure of which is hereby incorporated by reference in its entirety.

Once the samples are introduced into their respective partitions, in accordance with the methods and systems described herein, the sample nucleic acids within partitions are generally provided with unique identifiers such that, upon characterization of those nucleic acids they may be attributed as having been derived from their respective origins. Accordingly, the sample nucleic acids can be co-partitioned with the unique identifiers (e.g., barcode sequences). In some aspects, the unique identifiers are provided in the form of oligonucleotides that comprise nucleic acid barcode sequences that may be attached to those samples. The oligonucleotides are partitioned such that as between oligonucleotides in a given partition, the nucleic acid barcode sequences contained therein are the same, but as between different partitions, the oligonucleotides can have differing barcode sequences. In some aspects, only one nucleic acid barcode sequence may be associated with a given partition, although in some cases, two or more different barcode sequences may be present.

The nucleic acid barcode sequences can include from 6 to about 20 or more nucleotides within the sequence of the oligonucleotides. These nucleotides may be completely contiguous, i.e., in a single stretch of adjacent nucleotides, or they may be separated into two or more separate subsequences that are separated by one or more nucleotides. In some cases, separated subsequences may be from about 4 to about 16 nucleotides in length.

The co-partitioned oligonucleotides can also comprise other functional sequences useful in the processing of the partitioned nucleic acids. These sequences include, e.g., targeted or random/universal amplification primer sequences for amplifying the genomic DNA from the individual nucleic acids within the partitions while attaching the associated barcode sequences, sequencing primers, hybridization or probing sequences, e.g., for identification of presence of the sequences, or for pulling down barcoded nucleic acids, or any of a number of other potential functional sequences. Again, co-partitioning of oligonucleotides and associated barcodes and other functional sequences, along with sample materials is described in, for example, U.S. Provisional Patent Application Nos. 61/940,318, filed Feb. 7, 2014, 61/991,018, Filed May 9, 2014, and U.S. patent application Ser. No. 14/316,383, filed on Jun. 26, 2014, as well as U.S. patent application Ser. No. 14/175,935, filed Feb. 7, 2014, the full disclosures of which is hereby incorporated by reference in their entireties.

Briefly, in one exemplary process, beads are provided that each may include large numbers of the above described oligonucleotides releasably attached to the beads, where all of the oligonucleotides attached to a particular bead may include the same nucleic acid barcode sequence, but where a large number of diverse barcode sequences may be represented across the population of beads used. In some cases, the population of beads may provide a diverse barcode sequence library that may include at least 1000 different barcode sequences, at least 10,000 different barcode sequences, at least 100,000 different barcode sequences, or in some cases, at least 1,000,000 different barcode sequences. Additionally, each bead may be provided with large numbers of oligonucleotide molecules attached. In particular, the number of molecules of oligonucleotides including the barcode sequence on an individual bead may be at least bout 10,000 oligonucleotides, at least 100,000 oligonucleotide molecules, at least 1,000,000 oligonucleotide molecules, at least 100,000,000 oligonucleotide molecules, and in some cases at least 1 billion oligonucleotide molecules.

The oligonucleotides may be releasable from the beads upon the application of a particular stimulus to the beads. In some cases, the stimulus may be a photo-stimulus, e.g., through cleavage of a photo-labile linkage that may release the oligonucleotides. In some cases, a thermal stimulus may be used, where elevation of the temperature of the beads environment may result in cleavage of a linkage or other release of the oligonucleotides form the beads. In some cases, a chemical stimulus may be used that cleaves a linkage of the oligonucleotides to the beads, or otherwise may result in release of the oligonucleotides from the beads.

In accordance with the methods and systems described herein, the beads including the attached oligonucleotides may be co-partitioned with the individual samples, such that a single bead and a single sample are contained within an individual partition. In some cases, where single bead partitions are desired, the relative flow rates of the fluids can be controlled such that, on average, the partitions contain less than one bead per partition, in order to ensure that those partitions that are occupied, are primarily singly occupied. Likewise, one may wish to control the flow rate to provide that a higher percentage of partitions are occupied, e.g., allowing for only a small percentage of unoccupied partitions. In some aspects, the flows and channel architectures are controlled as to ensure a desired number of singly occupied partitions, less than a certain level of unoccupied partitions and less than a certain level of multiply occupied partitions.

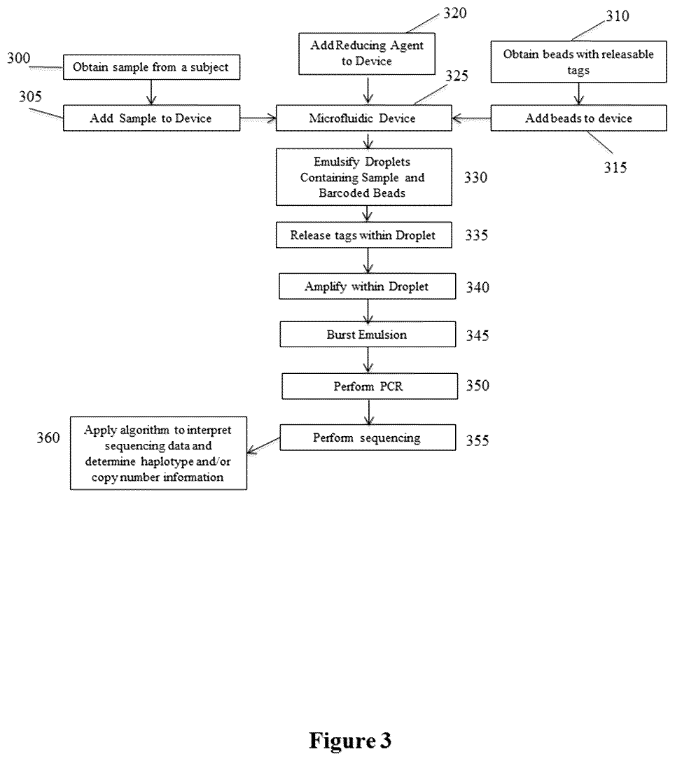

FIG. 3 illustrates an example method for barcoding and subsequently sequencing a sample nucleic acid, such as for use for a copy number variation or haplotype assay. First, a sample comprising nucleic acid may be obtained from a source, 300, and a set of barcoded beads may also be obtained, 310. The beads can be linked to oligonucleotides containing one or more barcode sequences, as well as a primer, such as a random N-mer or other primer. In some cases, the barcode sequences are releasable from the barcoded beads, e.g., through cleavage of a linkage between the barcode and the bead or through degradation of the underlying bead to release the barcode, or a combination of the two. For example, in some aspects, the barcoded beads can be degraded or dissolved by an agent, such as a reducing agent to release the barcode sequences. In this example, a low quantity of the sample comprising nucleic acid, 305, barcoded beads, 315, and, in some cases, other reagents, e.g., a reducing agent, 320, are combined and subject to partitioning. By way of example, such partitioning may involve introducing the components to a droplet generation system, such as a microfluidic device, 325. With the aid of the microfluidic device 325, a water-in-oil emulsion 330 may be formed, where the emulsion contains aqueous droplets that contain sample nucleic acid, 305, reducing agent, 320, and barcoded beads, 315. The reducing agent may dissolve or degrade the barcoded beads, thereby releasing the oligonucleotides with the barcodes and random N-mers from the beads within the droplets, 335. The random N-mers may then prime different regions of the sample nucleic acid, resulting in amplified copies of the sample after amplification, where each copy is tagged with a barcode sequence, 340. In some cases, each droplet contains a set of oligonucleotides that contain identical barcode sequences and different random N-mer sequences. Subsequently, the emulsion is broken, 345 and additional sequences (e.g., sequences that aid in particular sequencing methods, additional barcodes, etc.) may be added, , via, for example, amplification methods, 350 (e.g., PCR). Sequencing may then be performed, 355, and an algorithm applied to interpret the sequencing data, 360. Sequencing algorithms are generally capable, for example, of performing analysis of barcodes to align sequencing reads and/or identify the sample from which a particular sequence read belongs.

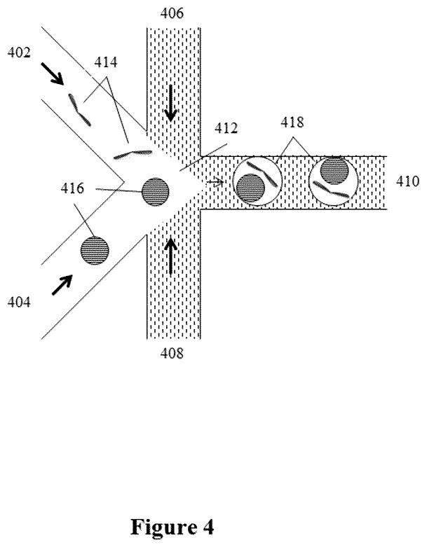

As noted above, while single bead occupancy may be desired, it will be appreciated that multiply occupied partitions, or unoccupied partitions may often be present. An example of a microfluidic channel structure for co-partitioning samples and beads comprising barcode oligonucleotides is schematically illustrated in FIG. 4. As shown, channel segments 402, 404, 406, 408 and 410 are provided in fluid communication at channel junction 412. An aqueous stream comprising the individual samples 414 is flowed through channel segment 402 toward channel junction 412. As described elsewhere herein, these samples may be suspended within an aqueous fluid prior to the partitioning process.

Concurrently, an aqueous stream comprising the barcode carrying beads 416 is flowed through channel segment 404 toward channel junction 412. A non-aqueous partitioning fluid is introduced into channel junction 412 from each of side channels 406 and 408, and the combined streams are flowed into outlet channel 410. Within channel junction 412, the two combined aqueous streams from channel segments 402 and 404 are combined, and partitioned into droplets 418, that include co-partitioned samples 414 and beads 416. As noted previously, by controlling the flow characteristics of each of the fluids combining at channel junction 412, as well as controlling the geometry of the channel junction, one can optimize the combination and partitioning to achieve a desired occupancy level of beads, samples or both, within the partitions 418 that are generated.

As will be appreciated, a number of other reagents may be co-partitioned along with the samples and beads, including, for example, chemical stimuli, nucleic acid extension, transcription, and/or amplification reagents such as polymerases, reverse transcriptases, nucleoside triphosphates or NTP analogues, primer sequences and additional cofactors such as divalent metal ions used in such reactions, ligation reaction reagents, such as ligase enzymes and ligation sequences, dyes, labels, or other tagging reagents.

Once co-partitioned, the oligonucleotides disposed upon the bead may be used to barcode and amplify the partitioned samples. An example process for use of these barcode oligonucleotides in amplifying and barcoding samples is described in detail in U.S. Patent Application Nos. 61/940,318, filed Feb. 7, 2014, 61/991,018, Filed May 9, 2014, and U.S. patent application Ser. No. 14/316,383, filed on Jun. 26, 2014, the full disclosures of which are hereby incorporated by reference in their entireties. Briefly, in one aspect, the oligonucleotides present on the beads that are co-partitioned with the samples and released from their beads into the partition with the samples. The oligonucleotides can include, along with the barcode sequence, a primer sequence at its 5′end. This primer sequence may be a random oligonucleotide sequence intended to randomly prime numerous different regions of the samples, or it may be a specific primer sequence targeted to prime upstream of a specific targeted region of the sample.

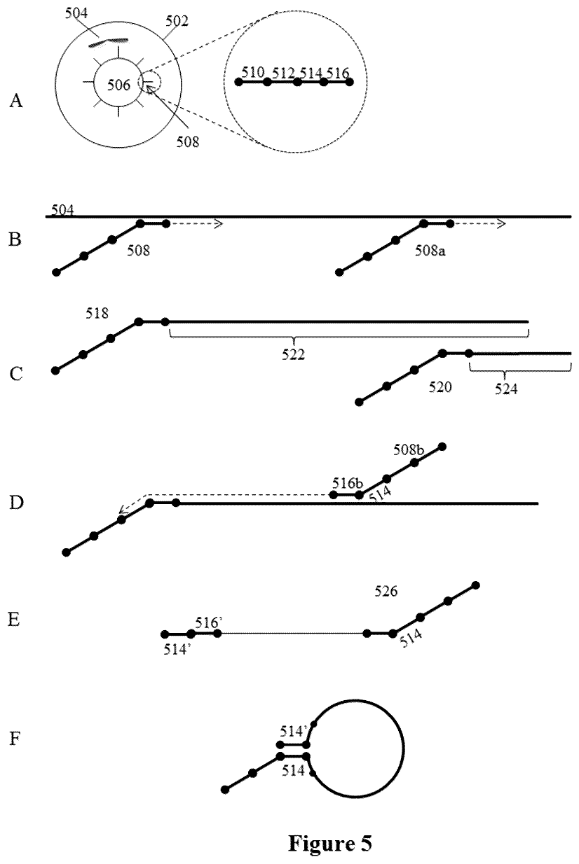

Once released, the primer portion of the oligonucleotide can anneal to a complementary region of the sample. Extension reaction reagents, e.g., DNA polymerase, nucleoside triphosphates, co-factors (e.g., Mg²⁺ or Mn²⁺ etc.), that are also co-partitioned with the samples and beads, then extend the primer sequence using the sample as a template, to produce a complementary fragment to the strand of the template to which the primer annealed, with complementary fragment includes the oligonucleotide and its associated barcode sequence. Annealing and extension of multiple primers to different portions of the sample may result in a large pool of overlapping complementary fragments of the sample, each possessing its own barcode sequence indicative of the partition in which it was created. In some cases, these complementary fragments may themselves be used as a template primed by the oligonucleotides present in the partition to produce a complement of the complement that again, includes the barcode sequence. In some cases, this replication process is configured such that when the first complement is duplicated, it produces two complementary sequences at or near its termini, to allow the formation of a hairpin structure or partial hairpin structure, that reduces the ability of the molecule to be the basis for producing further iterative copies. A schematic illustration of one example of this is shown in FIG. 5.

As the figure shows, oligonucleotides that include a barcode sequence are co-partitioned in, e.g., a droplet 502 in an emulsion, along with a sample nucleic acid 504. As noted elsewhere herein, the oligonucleotides 508 may be provided on a bead 506 that is co-partitioned with the sample nucleic acid 504, which oligonucleotides can be releasable from the bead 506, as shown in panel A. The oligonucleotides 508 include a barcode sequence 512, in addition to one or more functional sequences, e.g., sequences 510, 514 and 516. For example, oligonucleotide 508 is shown as comprising barcode sequence 512, as well as sequence 510 that may function as an attachment or immobilization sequence for a given sequencing system, e.g., a P5 sequence used for attachment in flow cells of an Illumina Hiseq or Miseq system. As shown, the oligonucleotides also include a primer sequence 516, which may include a random or targeted N-mer for priming replication of portions of the sample nucleic acid 504. Also included within oligonucleotide 508 is a sequence 514 which may provide a sequencing priming region, such as a “read1” or R1 priming region, that is used to prime polymerase mediated, template directed sequencing by synthesis reactions in sequencing systems. In some cases, the barcode sequence 512, immobilization sequence 510 and R1 sequence 514 may be common to all of the oligonucleotides attached to a given bead. The primer sequence 516 may vary for random N-mer primers, or may be common to the oligonucleotides on a given bead for certain targeted applications.

Based upon the presence of primer sequence 516, the oligonucleotides are able to prime the sample nucleic acid as shown in panel B, which allows for extension of the oligonucleotides 508 and 508a using polymerase enzymes and other extension reagents also co-portioned with the bead 506 and sample nucleic acid 504. As shown in panel C, following extension of the oligonucleotides that, for random N-mer primers, would anneal to multiple different regions of the sample nucleic acid 504; multiple overlapping complements or fragments of the nucleic acid are created, e.g., fragments 518 and 520. Although including sequence portions that are complementary to portions of sample nucleic acid, e.g., sequences 522 and 524, these constructs are generally referred to herein as comprising fragments of the sample nucleic acid 504, having the attached barcode sequences. As will be appreciated, the replicated portions of the template sequences as described above are often referred to herein as “fragments” of that template sequence. Notwithstanding the foregoing, however, the term “fragment” encompasses any representation of a portion of the originating nucleic acid sequence, e.g., a template or sample nucleic acid, including those created by other mechanisms of providing portions of the template sequence, such as actual fragmentation of a given molecule of sequence, e.g., through enzymatic, chemical or mechanical fragmentation. In some aspects, however, fragments of a template or sample nucleic acid sequence may denote replicated portions of the underlying sequence or complements thereof.

The barcoded nucleic acid fragments may then be subjected to characterization, e.g., through sequence analysis, or they may be further amplified in the process, as shown in panel D. For example, additional oligonucleotides, e.g., oligonucleotide 508b, also released from bead 306, may prime the fragments 518 and 520. In particular, again, based upon the presence of the random N-mer primer 516b in oligonucleotide 508b (which in some cases can be different from other random N-mers in a given partition, e.g., primer sequence 516), the oligonucleotide anneals with the fragment 518, and is extended to create a complement 526 to at least a portion of fragment 518 which includes sequence 528, that comprises a duplicate of a portion of the sample nucleic acid sequence. Extension of the oligonucleotide 508b continues until it has replicated through the oligonucleotide portion 508 of fragment 518. As noted elsewhere herein, and as illustrated in panel D, the oligonucleotides may be configured to prompt a stop in the replication by the polymerase at a desired point, e.g., after replicating through sequences 516 and 514 of oligonucleotide 508 that is included within fragment 518. As described herein, this may be accomplished by different methods, including, for example, the incorporation of different nucleotides and/or nucleotide analogues that are not capable of being processed by the polymerase enzyme used. For example, this may include the inclusion of uracil containing nucleotides within the sequence region 512 to prevent a non-uracil tolerant polymerase to cease replication of that region. As a result a fragment 526 is created that includes the full-length oligonucleotide 508b at one end, including the barcode sequence 512, the attachment sequence 510, the R1 primer region 514, and the random N-mer sequence 516b. At the other end of the sequence can be included the complement 516′ to the random N-mer of the first oligonucleotide 508, as well as a complement to all or a portion of the R1 sequence, shown as sequence 514′. The R1 sequence 514 and its complement 514′ are then able to hybridize together to form a partial hairpin structure 528. As will be appreciated because the random N-mers differ among different oligonucleotides, these sequences and their complements would not be expected to participate in hairpin formation, e.g., sequence 516′, which is the complement to random N-mer 516, would not be expected to be complementary to random N-mer sequence 516b. This would not be the case for other applications, e.g., targeted primers, where the N-mers would be common among oligonucleotides within a given partition.

By forming these partial hairpin structures, it allows for the removal of first level duplicates of the sample sequence from further replication, e.g., preventing iterative copying of copies. The partial hairpin structure also provides a useful structure for subsequent processing of the created fragments, e.g., fragment 526.

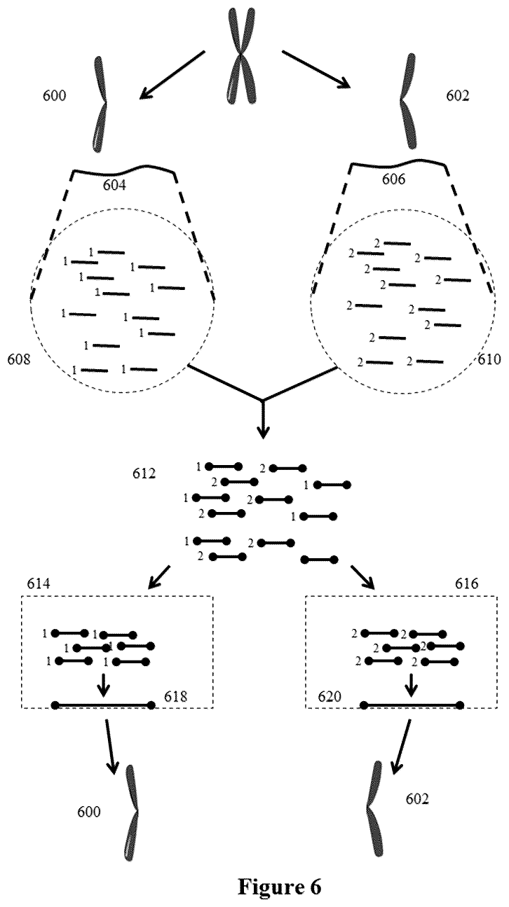

All of the fragments from multiple different partitions may then be pooled for sequencing on high throughput sequencers as described herein. Because each fragment is coded as to its partition of origin, the sequence of that fragment may be attributed back to its origin based upon the presence of the barcode. This is schematically illustrated in FIG. 6. As shown in one example, a nucleic acid 604 originated from a first source 600 (e.g., individual chromosome, strand of nucleic acid, etc.) and a nucleic acid 606 derived from a different chromosome 602 or strand of nucleic acid are each partitioned along with their own sets of barcode oligonucleotides as described above.

Within each partition, each nucleic acid 604 and 606 is then processed to separately provide overlapping set of second fragments of the first fragment(s), e.g., second fragment sets 608 and 610. This processing also provides the second fragments with a barcode sequence that is the same for each of the second fragments derived from a particular first fragment. As shown, the barcode sequence for second fragment set 608 is denoted by “1” while the barcode sequence for fragment set 610 is denoted by “2”. A diverse library of barcodes may be used to differentially barcode large numbers of different fragment sets. However, it is not necessary for every second fragment set from a different first fragment to be barcoded with different barcode sequences. In some cases, multiple different first fragments may be processed concurrently to include the same barcode sequence. Diverse barcode libraries are described in detail elsewhere herein.

The barcoded fragments, e.g., from fragment sets 608 and 610, may then be pooled for sequencing using, for example, sequence by synthesis technologies available from Illumina or Ion Torrent division of Thermo Fisher, Inc. Once sequenced, the sequence reads 612 can be attributed to their respective fragment set, e.g., as shown in aggregated reads 614 and 616, at least in part based upon the included barcodes, and in some cases, in part based upon the sequence of the fragment itself. The attributed sequence reads for each fragment set are then assembled to provide the assembled sequence for each sample fragment, e.g., sequences 618 and 620, which in turn, may be further attributed back to their respective original chromosomes (600 and 602). Methods and systems for assembling genomic sequences are described in, for example, U.S. Provisional Patent Application No. 62/017,589, filed Jun. 26, 2014, the full disclosure of which is hereby incorporated by reference in its entirety. In some examples, genomic sequences are assembled by de novo assembly and/or reference based assembly (e.g., mapping to a reference).

### III. Application of Methods and Systems to Phasing and Copy Number Assays

In one aspect of the systems and methods described herein, the ability to attribute sequence reads to longer originating molecules is used in determining phase information about the sequence. In one example, barcodes associated with sequences that reveal two or more specific gene variant sequences (e.g., alleles, genetic markers) are compared to determine whether or not that set of genetic markers reside on the same chromosome or different chromosomes in the sample. Such phasing information can be used in order to determine the relative copy number of certain target chromosomes or genes in a sample. An advantage of the described methods and symptoms is that multiple locations, loci, variants, etc. can be used to identify individual chromosomes or nucleic acid strands from which they originate in order to determine phasing and copy number information. Often, multiple locations (e.g., greater than 2, 3, 4, 5, 6, 7, 8, 9, 10, 20, 30, 40, 50, 100, 500, 1000, 5000, 10000, 50000, 100000, or 500000) along a chromosome are used in order to determine phasing, haplotype and copy number variation information described herein.

By way of example, as noted above, the methods and systems described herein, by virtue of the partitioning and attribution aspects described above, can be useful at providing effective long sequence reads from individual nucleic acid fragments, e.g., individual nucleic acid molecules, despite utilizing sequencing technology that may provide relatively shorter sequence reads. Because these long sequence reads may be attributed to single starting fragments or molecules, variant locations in the sequence can, likewise, be attributed to a single molecule, and by extrapolation, to a single chromosome. In addition, one may employ the multiple locations on any given fragment, as alignment features for adjacent fragments, to provide aligned sequences that can be inferred as originating from the same chromosome. By way of example, a first fragment may be sequenced, and by virtue of the attribution methods and systems described above, the variants present on that sequence may all be attributed to a single chromosome. A second fragment that shares a plurality of these variants that are determined to be present only on one chromosome, may then be assumed to be derived from the same chromosome, and thus aligned with the first, to create a phased alignment of the two fragments. Repeating this allows for the identification of long range phase information. Identification of variants on a single chromosome can be obtained from either known references, e.g., HapMap, or from an aggregation of the sequencing data, e.g., showing differing variants on an otherwise identical sequence stretch.

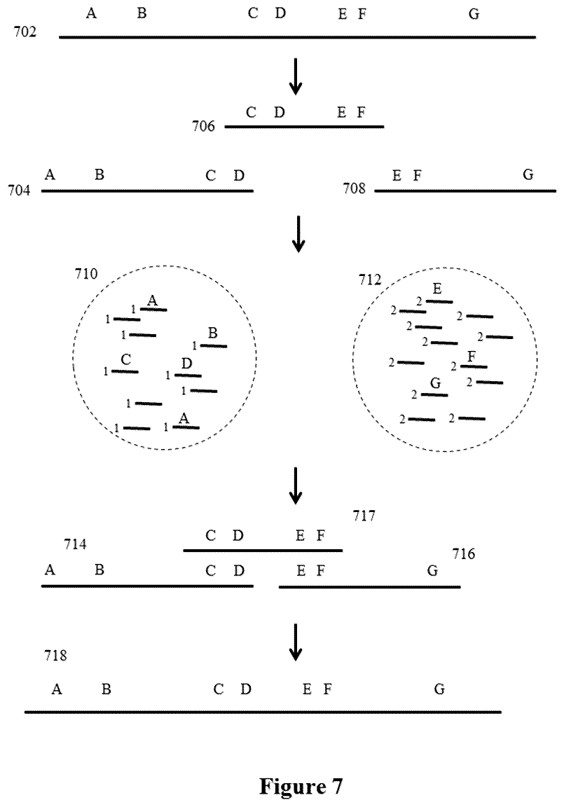

FIG. 7 provides a schematic illustration of an example phased sequencing process. As shown, an originating nucleic acid 702, such as, for example, a chromosome, a chromosome fragment, an exome, or other large, single nucleic acid molecule, can be fragmented into multiple large fragments 704, 706, 708. The originating nucleic acid 702 may include a number of sequence variants (A, B, C, D, E, F, and G) that are specific to the particular nucleic acid molecule, e.g., chromosome. In accordance with the processes described herein, the originating nucleic acid can be fragmented into multiple large, overlapping fragments 704, 706 and 708, that include subsets of the associated sequence variants. Each fragment can then be partitioned, further fragmented into subfragments, and barcoded, as described herein to provide multiple overlapping, barcoded subfragments of the larger fragments, where subfragments of a given larger fragment bear the same barcode sequence. For example, subfragments associated with barcode sequence “1” and barcode sequence “2” are shown in partitions 710 and 712, respectively, The barcoded subfragments can then be pooled, sequenced, and the sequenced subfragments assembled to provide long fragment sequences 714, 716, and 717. One or more of the long fragment sequences 714, 716, and 717 can include multiple variants. The long fragment sequences may then be further assembled, based upon overlapping phased variant information from sequences 714, 716, and 717 to provide a phased sequence 718, from which phased locations can be determined.

Once the phased locations are determined, one may further exploit that information in a variety of ways. For example, one can utilize knowledge of phased variants in assessing genetic risk for certain disorders, identify paternal vs. maternal characteristics, identify aneuploidies, or identify haplotyping information.

In some aspects of the systems and methods disclosed herein, copy number variation assays are performed using simultaneous detection of two or more phased genetic markers to improve the accuracy of copy number counting. Utilizing the phasing information can increase the relative strength of the signal compared to the variance under a naïve method just based on counting reads over multiple loci and across haplotypes. Additionally, utilizing phasing information allows for normalization of position-specific biases, boosting the signal substantially further. Copy number variation (CNV) accuracy may depend on myriad factors including sequencing depth, length of CNV, number of copies, etc). The methods and systems provided herein may determine CNV with an accuracy of at least 70%, 80%, 85%, 90%, 91%, 92%, 93%, 94%, 95%, 99%, 99.1%, 99.2%, 99.3% 99.4%, 99.5%, 99.6%, 99.7%, 99.8%, 99.9%, 99.95%, 99.99%, 99.995%, or 99.999%. In some cases, the methods and systems provided herein determine CNV with an error rate of less than 10%, 9%, 8%, 7%, 6%, 5%, 4%, 3%, 2%, 1%, 0.1%, 0.05%, 0.01%, 0.005%, 0.001%, 0.0005%, 0.0001%, 0.00005%, 0.00001%, or 0.000005%. Similarly, the methods and systems provided herein may detect phasing/haplotype information of two or more genetic variants with an accuracy of at least 70%, 80%, 85%, 90%, 91%, 92%, 93%, 94%, 95%, 99%, 99.1%, 99.2%, 99.3% 99.4%, 99.5%, 99.6%, 99.7%, 99.8%, 99.9%, 99.95%, 99.99%, 99.995%, or 99.999%. In some cases, the methods and systems provided herein determine phasing or haplotype information with an error rate of less than 10%, 9%, 8%, 7%, 6%, 5%, 4%, 3%, 2%, 1%, 0.1%, 0.05%, 0.01%, 0.005%, 0.001%, 0.0005%, 0.0001%, 0.00005%, 0.00001%, or 0.000005%. This disclosure also provides methods of removing locus-specific biases, where the locus-specific variance are reduced by at least 2-fold, 3-fold, 4-fold, 5-fold, 10-fold, 20-fold, 30-fold, 40-fold, 50-fold, 60-fold, 70-fold, 80-fold, 90-fold, 100-fold, 200-fold, 500-fold, 1000-fold, 5000-fold, or 10000-fold. The methods and systems provided herein can be used to detect variations in copy number, such as where the change in copy number reflects a change in the number of chromosomes, or portions of chromosomes. In some cases, the methods and systems provided herein can be used to detect variations in copy number of a gene present on the same chromosome.

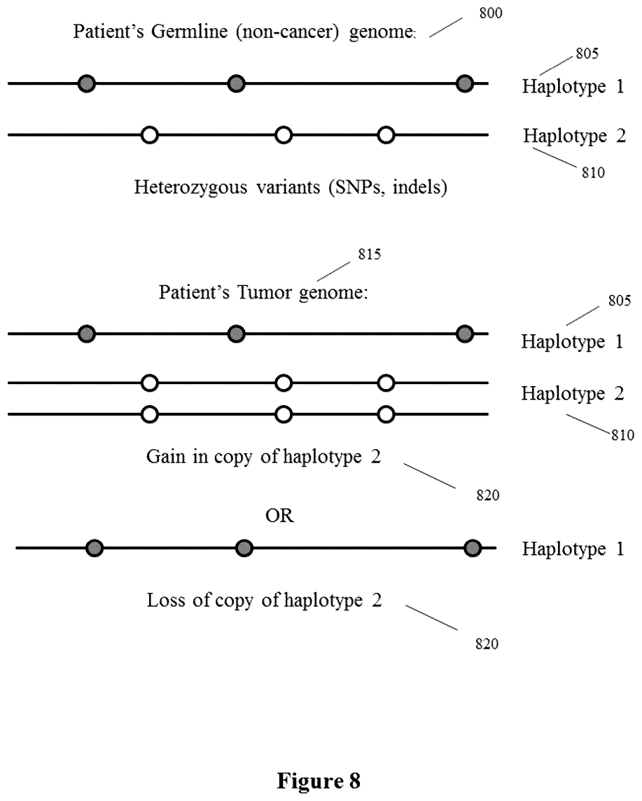

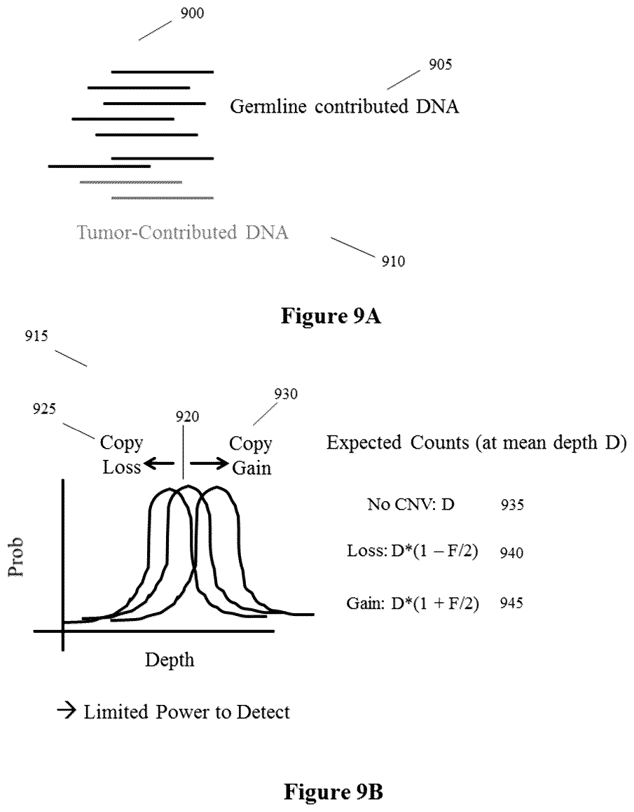

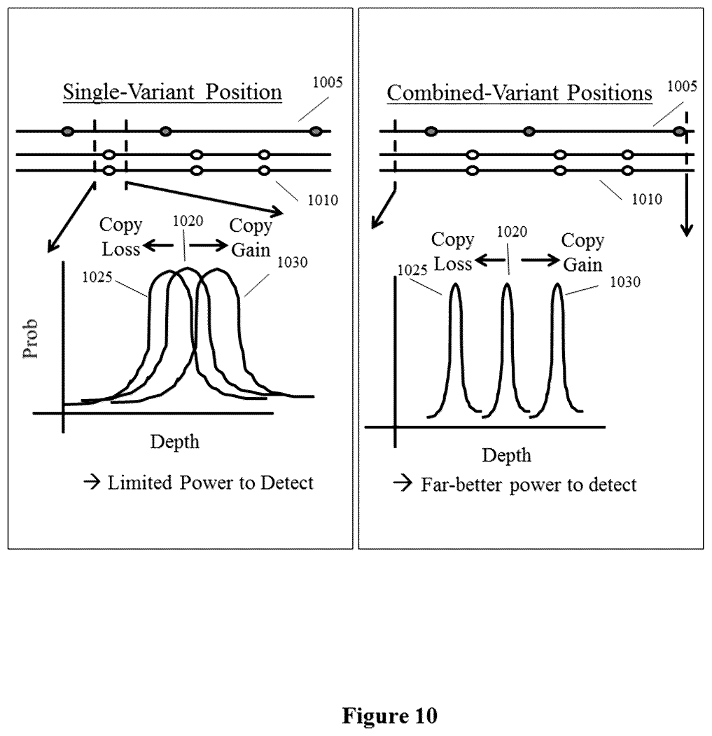

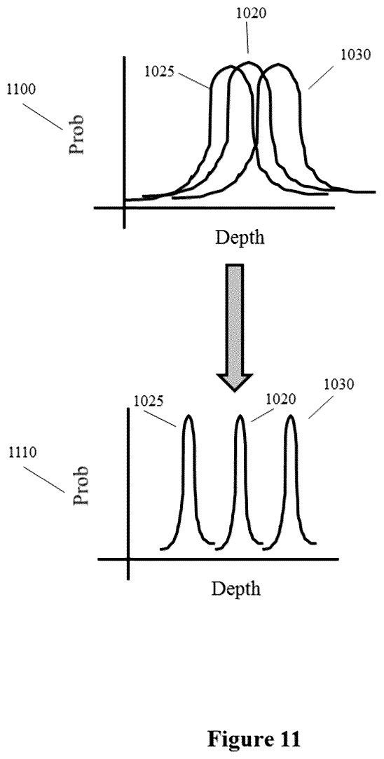

FIG. 8 (top panel) is a schematic illustrating a subset of a healthy patient's genome. This patient has a heterozygous genotype at the indicated loci and two separate haplotypes (1 and 2) 805, 810 located on separate chromosome strands. The patient's naturally-occurring variations (such as SNPs or indels) are depicted as circles. FIG. 8 also depicts the genome of a patient with cancer 815. Certain cancers are associated with a gain in haplotype copy number. The middle panel depicts a gain in a haplotype 2, 810. Cancers may also be associated with a loss in haplotype number, as depicted in the bottom panel of FIG. 8, which shows a loss of haplotype 2 820. Common sequencing techniques cannot accurately determine this loss or gain of haplotype copies. As shown in FIG. 9a this is in part due to the fact that the tumor-contributed DNA 910 in a patient's blood is only a small fraction of the total DNA, of which a majority is the DNA contributed by normal tissue 905. This low concentration of tumor DNA results in imprecise detection of copy number with normal sequencing techniques, see FIG. 9b. The difference in the peaks of expected counts at mean depth D 935 for no copy variation 920 and the peaks for copy loss 925 (940) and copy gain 930 (945) is difficult to detect. For any given individual marker, the distribution of results of the copy number assay in replicate testing can be distributed around the correct answer in a manner approximating a Poisson distribution, where the width of the distribution is dependent on various sources of random error in the assay. Since for a give sample the change in copy number may be relatively small portion of the sample, broad probability distributions for monitoring of single genetic markers can mask the correct result. This difficulty is due to the fact that normal sequencing techniques only look at one single variant position of a haplotype at a time, as shown in FIG. 10 (left panel). Using such techniques, there can be significant overlap between peaks representing copy loss 1025, normal copy 1020, and copy gain 1030. The techniques disclosed herein allow for detection of whole (or partial) haplotypes, increasing the resolution and improving the detection of copy gain and loss, FIG. 10 (right panel). This improvement is schematically shown in FIG. 11, where normal detection 1100 results in spread out, overlapping peaks while the techniques herein 1110 allow for finer peaks and improved resolution of copy gain or loss. The use of simultaneous monitoring of two or more phased genetic markers, particularly markers that are known to be co-located on a single chromosome, and which can therefore most likely always appear in greater or lesser number in a synchronized, non-random fashion has the effect of narrowing the width of the expected results distribution and simultaneously improving the accuracy of the count.

In addition to advantages in detecting and diagnosing cancers, the methods and systems provided herein also provide more accurate and sensitive processes for detecting fetal aneuploidy.

Fetal aneuploidies are aberrations in fetal chromosome number. Aneuploidies commonly result in significant physical and neurological impairments. For example, a reduction in the number of X chromosomes is responsible for Turner's syndrome. An increase in copy number of chromosome number 21 results in Down Syndrome. Invasive testing such as amniocentesis or Chorionic Villus Sampling (CVS) can lead to risk of pregnancy loss and less invasive methods of testing the maternal blood are used here.

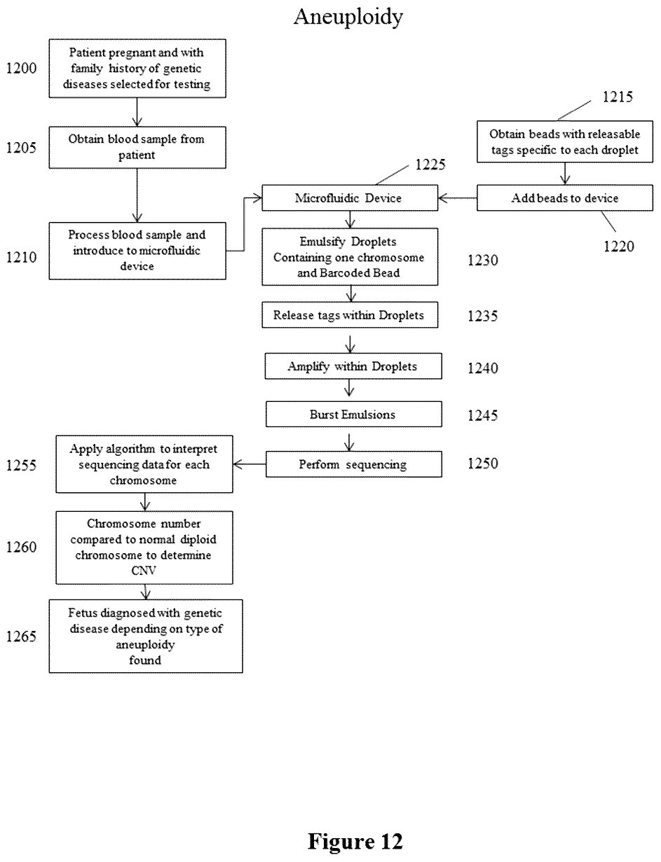

Methods described herein may be useful in non-invasively detecting fetal aneuploidies. An exemplary process is shown in FIG. 12. A pregnant woman at risk of carrying a fetus with an aneuploid genome is tested, 1200. A maternal blood sample containing fetal genetic material is collected, 1205. Genetic material (e.g., cell-free nucleic acids) is then extracted from the blood sample, 1210. A set of barcoded beads may also be obtained, 1215. The beads can be linked to oligonucleotides containing one or more barcode sequences, as well as a primer, such as a random N-mer or other primer. In some cases, the barcode sequences are releasable from the barcoded beads, e.g., through cleavage of a linkage between the barcode and the bead or through degradation of the underlying bead to release the barcode, or a combination of the two. For example, in some aspects, the barcoded beads can be degraded or dissolved by an agent, such as a reducing agent to release the barcode sequences. In this example, a sample, 1210, barcoded beads, 1220, and, in some cases, other reagents, e.g., a reducing agent, are combined and subjected to partitioning. By way of example, such partitioning may involve introducing the components to a droplet generation system, such as a microfluidic device, 1225. With the aid of the microfluidic device 1225, a water-in-oil emulsion 1230 may be formed, where the emulsion contains aqueous droplets that contain sample nucleic acid, 1210, barcoded beads, 1215, and, in some cases, a reducing agent. The reducing agent may dissolve or degrade the barcoded beads, thereby releasing the oligonucleotides with the barcodes and random N-mers from the beads within the droplets, 1235. The random N-mers may then prime different regions of the sample nucleic acid, resulting in amplified copies of the sample after amplification, where each copy is tagged with a barcode sequence, 1240. In some cases, each droplet contains a set of oligonucleotides that contain identical barcode sequences and different random N-mer sequences. In other embodiments, individual droplets comprise unique bar-code sequences; or, in some cases, a certain proportion of the total population of droplets has unique sequences. Subsequently, the emulsion is broken, 1245 and additional sequences (e.g., sequences that aid in particular sequencing methods, additional barcodes, etc.) may be added, via, for example, amplification methods (e.g., PCR). Sequencing may then be performed via any suitable type of sequencing platform (e.g., Illumina, Ion Torrent, Pacific Biosciences SMRT, Roche 454 sequencing SOLiD sequencing, etc.), 1250, and an algorithm applied to interpret the sequencing data, 1255. Sequencing algorithms are generally capable, for example, of performing analysis of barcodes to align sequencing reads and/or identify the sample from which a particular sequence read belongs. The aligned sequences may be further attributed to their respective genetic origins (e.g., chromosomes) based upon, the unique barcodes attached. The number of chromosome copies is then compared to that of a normal diploid chromosome, 1260. The patient is informed of any copy number aberrations for different chromosomes and the associated risks/disease, 1265.

Phasing, e.g. determining whether genetic variants are linked or reside on different chromosomes can provide useful information for a variety of applications. By way of example, phasing is useful for determining if certain translocations of a genome associated with diseases are present. Detection of such translocations can also allow for differential diagnosis and modified treatment. Determination of which alleles in a genome are linked can be useful for considering how genes are inherited.

It can often be useful to know the pattern of alleles, the haplotype, for each individual chromosome of a chromosome pair. For example, two copies of an inactivating mutation present on one chromosome may be of limited consequence, but could have significant effect if distributed between the two chromosomes, e.g., where neither chromosome supplies active gene product. These effects can be expressed e.g., with increased risk of disease or lack of response to certain medications.

### IV. Application of Methods and Systems to Identification/Characterization of Structural Variations

In other applications, the method and systems described herein are highly useful in obtaining the long range molecular sequence information for identification and characterization of a wide range of different genetic structural variations. As noted above, these variations include a wide variety of different variant events, including insertions, deletions, duplications, retrotransposons, translocations, inversions short and long tandem repeats, and the like. These structural variations are of significant scientific interest, as they are believed to be associated with a range of diverse genetic diseases.

Despite the interest in these variations, there are few effective and efficient methods of identifying and characterizing these structural variations. In part, this is because these variations are not characterized by the presence of abnormal sequence segments, but instead, involve and abnormal sequence context of what would be considered normal sequence segments, or simply missing sequence information. Because of their relatively short read lengths, most sequencing technologies are unable to provide significant context, and especially, long range sequence context, e.g., beyond their read lengths, for the sequence reads they produce, and thus lose the identification of these variations in the assembly process. The difficulties in identifying these variations is further complicated by the ensemble approach of these technologies in which many molecules, e.g., multiple chromosomes, are combined to yield a consensus sequence that may include genomic material that both includes and does not include the variation.

In the context of the presently described methods and systems, however, one can utilize short read sequencing technologies to derive long range sequence information that is attributable to individual originating nucleic acid molecules, and thus retain the long range sequence context of variant regions contained in whole or in part in those individual molecules.

As described above, the methods and systems described herein are capable of providing long range sequence information that is attributable to individual originating nucleic acid molecules, and further, in possessing this long range sequence information, inferring even longer range sequence context, through the comparing and overlapping of these longer sequence information. Such long range sequence information and/or inferred sequence context allows the identification and characterization numerous structural variations not easily identified using available techniques.

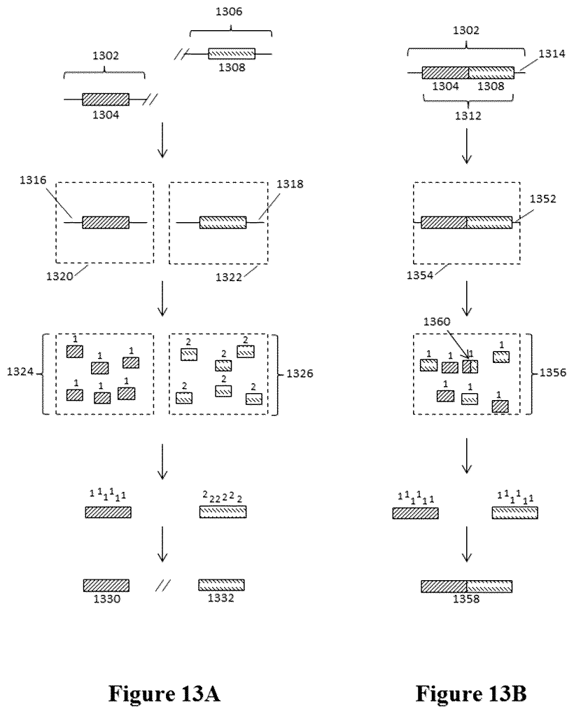

While illustrated in simplified fashion in FIG. 2, FIGS. 13A and 13B provide a more detailed example process for identifying certain types of structural variations using the methods and systems described herein. As shown, the genome of an organism, or tissue from an organism, might ordinarily include the first genotype illustrated in FIG. 13A, where a first gene region 1302 including first gene 1304 is separated from a second gene region 1306 including second gene 1208. This separation may reflect a range of distances between the genes, including, e.g., different regions in the same exon, different exons on the same chromosome, different chromosomes, etc. As shown in FIG. 13B however, a genotype is shown that reflects a translocation event having occurred in which gene 1308 is inserted into gene region 1304 such that it creates a gene fusion between genes 1304 and 1308 as gene fusion 1312 in variant sequence 1314.

Current methods for detecting large genomic structural variants (such as large inversions or translocations) rely on read pairs that span the breakpoints of the variants (for example the genomic loci where the translocated parts fused together). To ensure that such read pairs are observed during a sequencing experiment, very deep sequencing can be required. In targeted sequencing (such as exome sequencing), detecting structural variants with current sequencing technologies is almost impossible, unless the breakpoint is within the targeted regions (e.g. in an exon), which is very unlikely.

Information provided by the barcode methods and systems described herein, however, can greatly improve the ability to detect structural variants. Intuitively, the loci to the left and to the right of a breakpoint, can tend to be on a common fragment of genomic DNA and therefore be maintained within a single partition, and thus barcoded with a common or shared barcode sequence. Due to the stochastic nature of shearing, this sharing of barcodes decreases as the sequences are more distant from the breakpoint. Using statistical methods one can determine whether the barcode overlap between two genomic loci is significantly larger than what would be expected by chance. Such an overlap suggests the presence of a breakpoint. Importantly, the barcode information complements information provided by traditional sequencing (such as information from reads spanning the breakpoint) if such information is available.

In the context of the methods described herein, the genomic material from the organism, including the relevant gene regions is fragmented such that it includes relatively long fragments, as described above. This is illustrated with respect to the non-translocated genotype in FIG. 13A. As shown two long individual first molecule fragments 1316 and 1318 are created that include gene regions 1302 and 1306 respectively. These fragments are separately partitioned into partitions 1320 and 1322, respectively, and each of the first fragments is fragmented into a number of second fragments 1324 and 1326, respectively within the partition, which fragmenting process attaches a unique identifier tag or barcode sequence to the second fragments that is common to all of the second fragments within a given partition. The tag or barcode is indicated by “1” or “2”, for each of partitions 1320 and 1322, respectively. As a result, completely separate genes 1304 and 1308 can result in differently partitioned, and differently barcoded groups of second fragments.

Once barcoded, the second fragments may then be pooled and subjected to nucleic acid sequencing processes, which can provide both the sequence of the second fragment as well as the barcode sequence for that fragment. Based upon the presence of a particular barcode, e.g., 1 or 2, a the second fragment sequences may then be attributed to a certain originating sequence, e.g., gene 1304 or 1308, as shown by the attribution of barcodes to each sequence. In some cases, mapping of barcoded second fragment sequences as to separate originating first fragment sequences may be sufficiently definitive to determine that no translocation has occurred. However, in some cases, one may assemble the second fragment sequences to provide an assembled sequence for all or a portion of the originating first fragment sequence, e.g., as shown by assembled sequences 1330 and 1332.

In contrast to the non-translocated genotype example shown in FIG. 13A, FIG. 13B shows a schematic illustration of the same process applied to a translocation containing genotype. As shown, a first long nucleic acid fragment 1352 is generated from the variant sequence 1314, and includes at least a portion of the translocation variant, e.g., gene fusion 1312. The first fragment 1352 is then partitioned into discrete partition 1354. Within partition 1354, first fragment 1352 is further fragmented into second fragments 1356 that again, include unique barcodes that are the same for all second fragments 1356 within the partition 1354 (shown as barcode “1”). As above, pooling the second fragments and sequencing provides the underlying sequences of the second fragments as well as their associated barcodes. These barcoded sequences can then be attributed to their respective gene sequences. As shown, however, both genes can reflect attributed second fragment sequences that include the same barcode sequences, indicating that they originated from the same partition, and potentially the same originating molecule, indicating a gene fusion. This may be further validated by providing a number of overlapping first fragments that also include at least portions of the gene fusion, but processed in different partitions with different barcodes.

In some cases, the presence of multiple different barcode sequences (and their underlying fragment sequences) that attribute to each of the originally separated genes can be indicative of the presence of a gene fusion or other translocation event. In some cases, attribution of at least 2 barcodes, at least 3 different barcodes, at least 4 different barcodes, at least 5 different barcodes, at least 10 different barcodes, at least 20 different barcodes or more, to two genetic regions that would have been expected to have been separated based upon a reference sequence, may provide indication of a translocation event that has placed those regions proximal to, adjacent to or otherwise integrated with each other. In some cases, the size of the fragments that are partitioned can indicate the sensitivity with which one can identify variant linkage. In particular, where the fragments in a given droplet are 10 kb in length, it would be expected that linkages that are within that 10 kb size range would be detectable.

Likewise, where both the variant and the wild type structure fall within the same 10 kb fragment, it would be expected that identification of that variant would be more difficult, as both would show linkage through common or shared barcodes. As such, fragment size selection may be used to adjust the relative proximity of detected linked sequences, whether as wild type or variants. In general, however, structural variants that result in proximal sequences that are normally separated by more than 100 bases, more than 500 bases, more than 1 kb, 10 kb, more than 20 kb, more than 30 kb, more than 40 kb, more than 50 kb, more than 60 kb, more than 70 kb, more than 80 kb, more than 90 kb, more than 100 kb, more than 200 kb or even greater, may be readily identified herein by identifying the linkage between those unlinked sequence segments in variant genomes, which linkage is indicated by shared or common barcodes, and/or, as noted, by sequence data that spans a breakpoint. Such linkage is generally identifiable when those linked sequences are separated within the genomic sequence by less than 50 kb, less than 40 kb, less than 30 kb, less than 20 kb, less than 10 kb, less than 5 kb, less than 4 kb, less than 3 kb, less than 2 kb, less than 1 kb, less than 500 bases, less than 200 bases or even less.

In some cases, a structural variation resulting in two sequences being positioned proximal to each other or linked, where they would normally be separated by, e.g., more than 10 kb, more than 20 kb, more than 30 kb, more than 40 kb, or more than 50 kb or more, may be identified by the percentage of the total number of mappable barcoded sequences that include barcodes that are common to the two sequence portions.

As will be appreciated, in some cases, the processes described herein can ensure that sequences that are within a certain sequence distance will be included, whether as wild type or variant sequences, within a single partition, e.g., as a single nucleic acid fragment. For example, where common or overlapping barcode sequences are greater than 1% of the total number of barcodes mapped to the two sequences, it may be used to identify linkage as between two sequence segments, and particularly, as between two sequence segments that would not normally be linked, e.g., a structural variation. In some cases, the shared or common barcodes can be more than 2%, more than 3%, more than 4%, more than 5%, more than 6%, more than 7%, more than 8%, and in some cases more than 9% or even more than 10% of the total mappable barcodes to two normally separated sequences, in order to identify a structural linkage that constitutes a structural variation within the genome. In some cases, the shared or common barcodes can be detected at a proportion or number that is statistically significantly greater than a control genome that is known not to have the structural variation. Additionally, where second sequence fragments span the point where the variant sequence meets the “normal” sequence, or “breakpoint”, e.g., as in second fragment 1358 one can use this information as additional evidence of the gene fusion.

Again, as above, one can further elucidate the structure of the gene fusion 1312, by assembling the second fragment sequences to yield the assembled sequence of the gene fusion 1312, shown as assembled sequence 1360.

Further, while the presence of the barcode sequences allows the assembly of the short sequences into sequences for the longer originating fragments, these longer fragments also permit the inference of longer range sequence information from overlapping long fragments assembled from different, overlapping originating long fragments. This resulting assembly allows for longer range sequence level identification and characterization of gene fusion 1312.

In some cases, the methods described above are useful in identifying the presence of retrotransposons. Retrotransposons can be created by transcription followed by reverse transcription of spliced messenger RNA (mRNA) and insertion into a new location in the genome. Hence, these structural variants lack introns and are often interchromosomal but otherwise have diverse features. When retrotransposons introduce functional copies of genes they are referred to as retrogenes, which have been reported in human and Drosophila genomes. In other cases, retrocopies may contain the entire transcript, specific transcript isoforms or an incomplete transcript. In addition, alternative transcription start sites and promoter sequences sometimes reside within a transcript so retrotransposons sometimes introduce promotor sequences within the reinserted region of the genome that could drive expression of downstream sequences.

Unlike tandem duplications, retrotransposons insert far away from the parental gene within exons or introns. When inserted near genes retrotransposons can exploit local regulatory sequences for expression. Insertions near genes can also inactivate the receiving gene or create new chimera transcripts. Retrotransposon mediated chimeric gene transcripts have been reported in RNA-Seq data from human samples.

Despite the significance of retrotransposons their detection can be limited to directed approaches relying on paired read support from mate pair libraries, exon-exon junction discovery in whole genome sequencing (WGS) or RNA-Seq recognition of retrotransposon chimeras. All of these methods can have false positives that complicate analysis.

Retrotransposons can be identified from whole genome libraries using the systems and methods described herein, and their insertion site can be mapped using the barcode mapping discussed above. For example, the Ceph NA12878 genome has a SKA3-DDX10 chimeric retrotransposon. The SKA3 intron-less transcript is inserted in between exons 10 and 11 of DDX10. Furthermore the CBX3-C150RF17 retrotransposon can also be detected in NA12878 using the methods described herein. Isoform 2 of CBX3 is inserted in between exons 2 and 3 of C150RF17. This chimeric transcript has been observed in 20% of European RNA-Seq samples from the HapMap project (D. R. Schrider et al. PLoS Genetics 2013).

Retrotransposons can also be detected in whole exome libraries prepared using the methods and systems described herein. While retrotransposons are easily enriched with exome targeting it can be difficult or not possible to differentiate between a translocation event and a retrotransposon since introns are removed during capture. However, using the systems and methods described herein, one may identify retrotransposons in whole exome sequencing (WES) libraries by introducing intronic baits for suspected retrotransposons (see also U.S. Provisional Patent Application No. 62/072,164, filed Oct. 29, 2014, incorporated herein by reference in its entirety for all purposes). Lack of intron signal can be indicative of retrotransposon structural variants whereas intron signal can be indicative of a translocation.

As will be appreciated, the ability to use longer range sequence context in identifying and characterizing of the above-described variations is equally applicable to identifying the range of other structural variations, including insertions, deletion, retrotransposons, inversions, etc., by mapping barcodes to regions within the variation, and/or spanning the variation.

V. Diseases & Disorders Arising from Copy Number Variation

The present methods and systems provide a highly accurate and sensitive approach to diagnosing and/or detecting a wide range of diseases and disorders. Diseases associated with copy number variations can include, for example, DiGeorge/velocardiofacial syndrome (22q11.2 deletion), Prader-Willi syndrome (15q 11-q13 deletion), Williams-Beuren syndrome (7q1 1.23 deletion), Miller-Dieker syndrome (MDLS) (17p13.3 microdeletion), Smith-Magenis syndrome (SMS) (17p11.2 microdeletion), Neurofibromatosis Type 1 (NF1) (17q11.2 microdeletion), Phelan-McErmid Syndrome (22q13 deletion), Rett syndrome (loss-of-function mutations in MECp2 on chromosome Xq28), Merzbacher disease (CNV of PLP1), spinal muscular atrophy (SMA) (homozygous absence of telomerec SMN1 on chromosome 5q13), Potocki-Lupski Syndrome (PTLS, duplication of chromosome 17p.11.2). Additional copies of the PMP22 gene can be associated with Charcot-Marie-Tooth neuropathy type IA (CMT1A) and hereditary neuropathy with liability to pressure palsies (HNPP). The disease can be a disease described in Lupski J. (2007) Nature Genetics 39: S43-S47.

The methods and systems provided herein can also accurately detect or diagnose a wide range of fetal aneuploidies. Often, the methods provided herein comprise analyzing a sample (e.g., blood sample) taken from a pregnant woman in order to evaluate the fetal nucleic acids within the sample. Fetal aneuploidies, can include, e.g., trisomy 13 (Patau syndrome), trisomy 18 (Edwards syndrome), trisomy 21 (Down Syndrome), Klinefelter Syndrome (XXY), monosomy of one or more chromosomes (X chromosome monosomy, Turner's syndrome), trisomy X, trisomy of one or more chromosomes, tetrasomy or pentasomy of one or more chromosomes (e.g., XXXX, XXYY, XXXY, XYYY, XXXXX, XXXXY, XXXYY, XYYYY and XXYYY), triploidy (three of every chromosome, e.g. 69 chromosomes in humans), tetraploidy (four of every chromosome, e.g. 92 chromosomes in humans), and multiploidy. In some embodiments, an aneuploidy can be a segmental aneuploidy. Segmental aneuploidies can include, e.g., 1p36 duplication, dup(17)(p11.2p11.2) syndrome, Down syndrome, Pelizaeus-Merzbacher disease, dup(22)(q11.2q11.2) syndrome, and cat-eye syndrome. In some cases, an abnormal genotype, e.g., fetal genotype, is due to one or more deletions of sex or autosomal chromosomes, which can result in a condition such as Cri-du-chat syndrome, Wolf-Hirschhorn, Williams-Beuren syndrome, Charcot-Marie-Tooth disease, Hereditary neuropathy with liability to pressure palsies, Smith-Magenis syndrome, Neurofibromatosis, Alagille syndrome, Velocardiofacial syndrome, DiGeorge syndrome, Steroid sulfatase deficiency, Kallmann syndrome, Microphthalmia with linear skin defects, Adrenal hypoplasia, Glycerol kinase deficiency, Pelizaeus-Merzbacher disease, Testis-determining factor on Y, Azospermia (factor a), Azospermia (factor b), Azospermia (factor c), or 1p36 deletion. In some embodiments, a decrease in chromosomal number results in an XO syndrome

Excessive genomic DNA copy number variation is also associated with Li-Fraumeni cancer predisposition syndrome (Shlien et al. (2008) PNAS 105:11264-9). CNV is associated with malformation syndromes, including CHARGE (coloboma, heart anomaly, choanal atresia, retardation, genital, and ear anomalies), Peters-Plus, Pitt-Hopkins, and thrombocytopenia-absent radius syndrome (see e.g., Ropers HH (2007) Am J of Hum Genetics 81: 199-207). The relationship between copy number variations and cancer is described, e.g., in Shlien A. and Malkin D. (2009) Genome Med. 1(6): 62. Copy number variations are associated with, e.g., autism, schizophrenia, and idiopathic learning disability. See e.g., Sebat J., et al. (2007) Science 316: 445-9; Pinto J. et al.

As described herein, the methods and systems provided herein are also useful to detect CNVs associated with different types of cancer. For example, the methods and systems can be used to detect EGFR copy number, which can be increased in non-small cell lung cancer.

The methods and systems provided herein can also be used to determine a subject's level of susceptibility to a particular disease or disorder, including susceptibility to infection from a pathogen (e.g., viral, bacterial, microbial, fungal, etc.). For example, the methods can be used to determine a subject's susceptibility to HIV infection by analyzing the copy number of CCL3L1, given that a relatively high level of CCL3L1 is associated with lower susceptibility to HIV infection (Gonzalez E. et al. (2005) Science 307: 1434-1440). In another example, the methods can be used to determine a subject's susceptibility to system lupus erythematosus. In such cases, for example, the methods can be used to detect copy number of FCGR3B (CD16 cell surface immunoglobulin receptor) since a low copy number of this molecule is associated with an increased susceptibility to systemic lupus erythematosus (Aitman T. J. et al. (2006) Nature 439: 851-855). The methods and systems provided herein can also be used to detect CNVs associated with other diseases or disorders, such as CNVs associated with autism, schizophrenia, or idiopathic learning disability (Kinght et al., (1999) The Lancet 354 (9191): 1676-81.). Similarly, the methods and systems can be used to detect autosomal-dominant microtia, which is linked to five tandem copies of a copy-number-variable region at chromosome 4p16 (Balikova I. (2008) Am J. Hum Genet. 82: 181-187).

### VI. Detection, Diagnosis and Treatment of Diseases and Disorders

The methods and systems provided herein can also assist with the detection, diagnosis, and treatment of a disease or disorder. In some cases, a method comprises detecting a disease or disorder using a system or method described herein and further providing a treatment to a subject based on the detection of the disease. For example, if a cancer is detected, the subject may be treated by a surgical intervention, by administering a drug designed to treat such cancer, by providing a hormonal therapy, and/or by administering radiation or more generalized chemotherapy.

Often, the methods and systems also permit a differential diagnosis and may further comprise treating a patient with a targeted therapy. In general, differential diagnosis of a disease or disorder (or absence thereof) can be achieved by determining and characterizing a sequence of a sample nucleic acid obtained from a subject suspected of having the disease or disorder and further characterizing the sample nucleic acid as indicative of a disorder or disease state (or absence thereof) by comparing it to a sequence and/or sequence characterization of a reference nucleic acid indicative of the presence (or absence) of the disorder or disease state.

The reference nucleic acid sequence may be derived from a genome that is indicative of an absence of a disease or disorder state (e.g., germline nucleic acid) or may be derived from a genome that is indicative of a disease or disorder state (e.g., cancer nucleic acid, nucleic acid indicative of an aneuploidy, etc.). Moreover, the reference nucleic acid sequence (e.g., having lengths of longer than 1 kb, longer than 5 kb, longer than 10 kb, longer than 15 kb, longer than 20 kb, longer than 30 kb, longer than 40 kb, longer than 50 kb, longer than 60 kb, longer than 70 kb, longer than 80 kb, longer than 90 kb or even longer than 100 kb) may be characterized in one or more respects, with non-limiting examples that include determining the presence (or absence) of a particular sequence, determining the presence (or absence) of a particular haplotype, determining the presence (or absence) of one or more genetic variations (e.g., structural variations (e.g., a copy number variation, an insertion, a deletion, a translocation, an inversion, a retrotransposon, a rearrangement, a repeat expansion, a duplication, etc.), single nucleotide polymorphisms (SNPs), etc.) and combinations thereof Moreover, any suitable type and number of sequence characteristics of the reference sequence can be used to characterize the sequence of the sample nucleic acid. For example, one or more genetic variations (or lack thereof) or structural variations (or lack thereof) of a reference nucleic acid sequence may be used as a sequence signature to identify the reference nucleic acid as indicative of the presence (or absence) of a disorder or disease state. Based on the characterization of the reference nucleic acid sequence utilized, the sample nucleic acid sequence can be characterized in a similar manner and further characterized/identified as derived (or not derived) from a nucleic acid indicative of the disorder or disease based upon whether or not it displays a similar character to the reference nucleic acid sequence. In some cases, characterizations of sample nucleic acid sequence and/or the reference nucleic acid sequence and their comparisons may be completed with the aid of a programmed computer processor. In some cases, such a programmed computer processor can be included in a computer control system, such as in an example computer control system described elsewhere herein.

The sample nucleic acid may be obtained from any suitable source, including sample sources and biological sample sources described elsewhere herein. In some cases, the sample nucleic acid may comprise cell-free nucleic acid. In some cases, the sample nucleic acid may comprise tumor nucleic acid (e.g., tumor DNA). In some cases, the sample nucleic acid may comprise circulating tumor nucleic acid (e.g., circulating tumor DNA (ctDNA)). Circulating tumor nucleic acid may be derived from a circulating tumor cell (CTC) and/or may be obtained, for example, from a subject's blood, plasma, other bodily fluid or tissue.

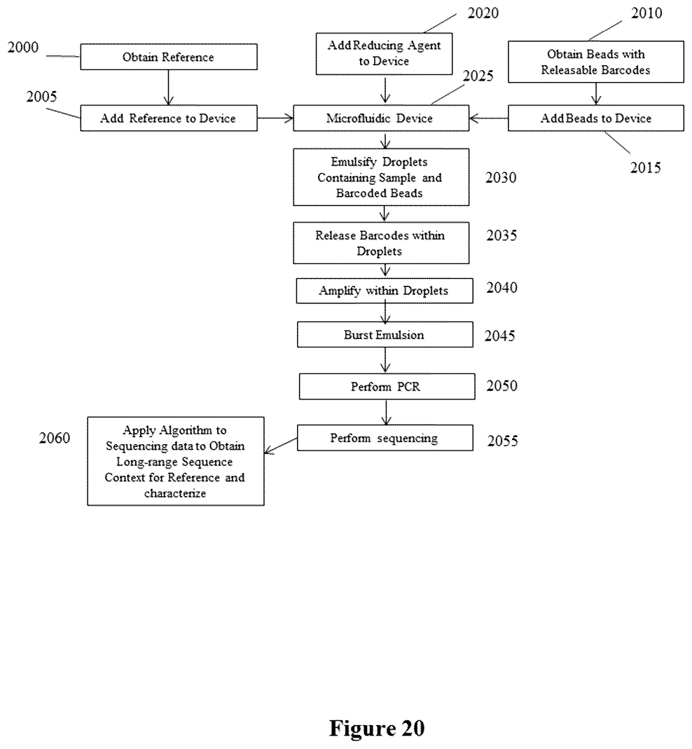

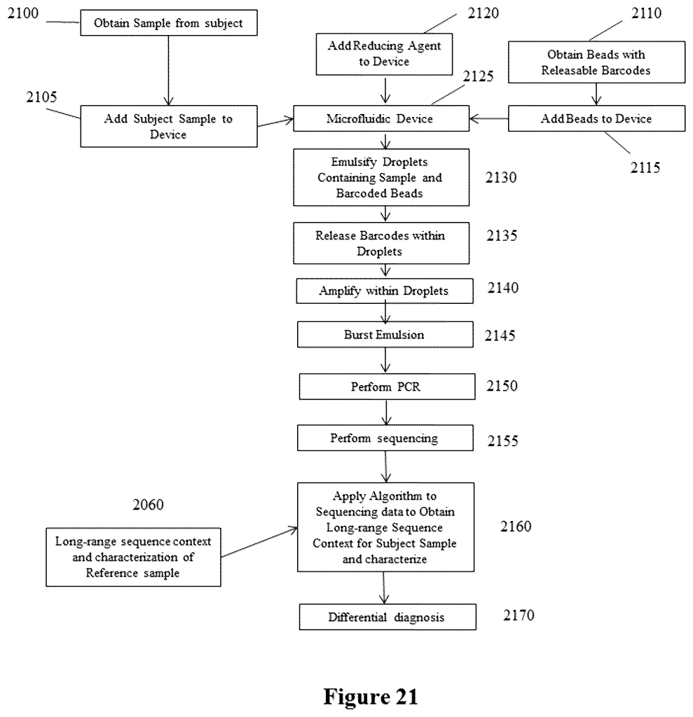

FIGS. 20-21 illustrate an example method for characterizing a sample nucleic acid in the context of disease detection and diagnosis. FIG. 20 demonstrates an example method by which long range sequence context can be determined for a reference nucleic acid (e.g., germline nucleic acid (e.g., germline genomic DNA), nucleic acid associated with a particular disorder or disease state) from shorter barcoded fragments, such as, for example in a manner analogous to that shown in FIG. 6. With respect to FIG. 20, a reference nucleic acid may be obtained 2000, and a set of barcoded beads may also be obtained, 2010. The beads can be linked to oligonucleotides containing one or more barcode sequences, as well as a primer, such as a random N-mer or other primer. In some cases, the barcode sequences are releasable from the barcoded beads, e.g., through cleavage of a linkage between the barcode and the bead or through degradation of the underlying bead to release the barcode, or a combination of the two. For example, in some aspects, the barcoded beads can be degraded or dissolved by an agent, such as a reducing agent to release the barcode sequences. In this example, reference nucleic acid, 2005, barcoded beads, 2015, and, in some cases, other reagents, e.g., a reducing agent, 2020, are combined and subject to partitioning. In some cases, the reference nucleic acid 2000 may be fragmented prior to partitioning and at least some of the resulting fragments are partitioned as 2005 for barcoding. By way of example, such partitioning may involve introducing the components to a droplet generation system, such as a microfluidic device, 2025. With the aid of the microfluidic device 2025, a water-in-oil emulsion 2030 may be formed, where the emulsion contains aqueous droplets that contain reference nucleic acid, 2005, reducing agent, 2020, and barcoded beads, 2015. The reducing agent may dissolve or degrade the barcoded beads, thereby releasing the oligonucleotides with the barcodes and random N-mers from the beads within the droplets, 2035. The random N-mers may then prime different regions of the reference nucleic acid, resulting in amplified copies of the reference nucleic acid after amplification, where each copy is tagged with a barcode sequence, 2040. In some cases, amplification 2040 may be achieved by a method analogous to that described elsewhere herein and schematically depicted in FIG. 5. In some cases, each droplet contains a set of oligonucleotides that contain identical barcode sequences and different random N-mer sequences. Subsequently, the emulsion is broken, 2045 and additional sequences (e.g., sequences that aid in particular sequencing methods, additional barcodes, etc.) may be added, via, for example, amplification methods, 2050 (e.g., PCR). Sequencing may then be performed, 2055, and an algorithm applied to interpret the sequencing data, 2060. In some cases, interpretation of the sequencing data 2060 may include providing a sequence for at least a portion of the reference nucleic acid. In some cases, long range sequence context for the reference nucleic acid is obtained and characterized such as, for example, in the case where the reference nucleic acid is derived from a disease state (e.g., determination of one or more haplotypes as described elsewhere herein, determination of one or more structural variations (e.g., a copy number variation, an insertion, a deletion, a translocation, an inversion, a rearrangement, a repeat expansion, a duplication, retrotransposon, a gene fusion, etc.), calling of one or more SNPs, etc.). In some cases, variants can be called for various reference nucleic acids obtained from a source and inferred contigs generated to provide longer range sequence context, such as is described elsewhere herein with respect to FIG. 7.

FIG. 21 demonstrates an example of characterizing a sample nucleic acid sequence from the reference 2060 characterization obtained as shown in FIG. 20. Long range sequence context can be obtained for the sample nucleic acid from sequencing of shorter barcoded fragments as is described elsewhere herein, such as, for example, via the method schematically depicted in FIG. 6. As shown in FIG. 21, a nucleic acid sample (e.g., a sample comprising a circulating tumor nucleic acid) can be obtained from a subject suspected of having a disorder or disease (e.g., cancer) 2100 and a set of barcoded beads may also be obtained, 2110. The beads can be linked to oligonucleotides containing one or more barcode sequences, as well as a primer, such as a random N-mer or other primer. In some cases, the barcode sequences are releasable from the barcoded beads, e.g., through cleavage of a linkage between the barcode and the bead or through degradation of the underlying bead to release the barcode, or a combination of the two. For example, in some aspects, the barcoded beads can be degraded or dissolved by an agent, such as a reducing agent to release the barcode sequences. In this example, sample nucleic acid, 2105, barcoded beads, 2115, and, in some cases, other reagents, e.g., a reducing agent, 2120, are combined and subject to partitioning. In some cases, the fetal sample 2100 is fragmented prior to partitioning and at least some of the resulting fragments are partitioned as 2105 for barcoding. By way of example, such partitioning may involve introducing the components to a droplet generation system, such as a microfluidic device, 2125. With the aid of the microfluidic device 2125, a water-in-oil emulsion 2130 may be formed, where the emulsion contains aqueous droplets that contain sample nucleic acid, 2105, reducing agent, 2120, and barcoded beads, 2115. The reducing agent may dissolve or degrade the barcoded beads, thereby releasing the oligonucleotides with the barcodes and random N-mers from the beads within the droplets, 2135. The random N-mers may then prime different regions of the sample nucleic acid, resulting in amplified copies of the sample nucleic acid after amplification, where each copy is tagged with a barcode sequence, 2140. In some cases, amplification 2140 may be achieved by a method analogous to that described elsewhere herein and schematically depicted in FIG. 5. In some cases, each droplet contains a set of oligonucleotides that contain identical barcode sequences and different random N-mer sequences. Subsequently, the emulsion is broken, 2145 and additional sequences (e.g., sequences that aid in particular sequencing methods, additional barcodes, etc.) may be added, via, for example, amplification methods, 2150 (e.g., PCR). Sequencing may then be performed, 2155, and an algorithm applied to interpret the sequencing data, 2160. In some cases, interpretation of the sequencing data 2160 may include providing a sequence of the sample nucleic acid. In some cases, long range sequence context for the nucleic acid sample is obtained. The sample nucleic acid sequence can be characterized 2160 (e.g., determination of one or more haplotypes as described elsewhere herein, determination of one or more structural variations (e.g., a copy number variation, an insertion, a deletion, a translocation, an inversion, a rearrangement, a repeat expansion, a duplication, retrotransposon, a gene fusion, etc.) using the characterization of the reference nucleic acid sequence 2060. Based on the comparison of the sample nucleic acid sequence and its characterization with the sequence and characterization of the reference nucleic acid, a differential diagnosis 2170 regarding the presence (or absence) of the disorder or disease state can be made.

As can be appreciated, analysis of reference nucleic acids and sample nucleic acids may completed as separate partitioning analyses or may be completed as part of a single partitioning analysis. For example, sample and reference nucleic acids may be added to the same device and barcoded sample and reference fragments generated in droplets according to FIGS. 20 and 21, where an emulsion comprises the droplets for both types of nucleic acid. The emulsion can then be broken and the contents of the droplets pooled, further processed (e.g., bulk addition of additional sequences via PCR) and sequenced as described elsewhere herein. Individual sequencing reads from the barcoded fragments can be attributed to their respective sample sequence via barcode sequences. Sequences obtained from the sample nucleic acid can be characterized based upon the characterization of the reference nucleic acid sequence.

Utilizing methods and systems herein can improve accuracy in determining long range sequence context of nucleic acids, including the long-range sequence context of reference and sample nucleic acid sequences as described herein. The methods and systems provided herein may determine long-range sequence context of reference and/or sample nucleic acids with accuracy of at least 70%, 80%, 85%, 90%, 91%, 92%, 93%, 94%, 95%, 99%, 99.1%, 99.2%, 99.3% 99.4%, 99.5%, 99.6%, 99.7%, 99.8%, 99.9%, 99.95%, 99.99%, 99.995%, or 99.999%. In some cases, the methods and systems provided herein may determine long-range sequence context of reference and/or sample nucleic acids with an error rate of less than 10%, 9%, 8%, 7%, 6%, 5%, 4%, 3%, 2%, 1%, 0.1%, 0.05%, 0.01%, 0.005%, 0.001%, 0.0005%, 0.0001%, 0.00005%, 0.00001%, or 0.000005%.

Moreover, methods and systems herein can also improve accuracy in characterizing a reference nucleic acid sequence and/or sample nucleic acid sequence in one or more aspects (e.g., determination of a sequence, determination of one or more genetic variations, determination of haplotypes, etc.). Accordingly, the methods and systems provided herein may characterize a reference nucleic acid sequence and/or sample nucleic acid sequence in one or more aspects with an accuracy of at least 70%, 80%, 85%, 90%, 91%, 92%, 93%, 94%, 95%, 99%, 99.1%, 99.2%, 99.3% 99.4%, 99.5%, 99.6%, 99.7%, 99.8%, 99.9%, 99.95%, 99.99%, 99.995%, or 99.999%. In some cases, the methods and systems provided herein may characterize a reference nucleic acid sequence and/or sample nucleic acid sequence in one or more aspects with an error rate of less than 10%, 9%, 8%, 7%, 6%, 5%, 4%, 3%, 2%, 1%, 0.1%, 0.05%, 0.01%, 0.005%, 0.001%, 0.0005%, 0.0001%, 0.00005%, 0.00001%, or 0.000005%.

Moreover, as is discussed above, improved accuracy in determining long-range sequence context of reference nucleic acids and characterization of the same can result in improved accuracy in sequencing and characterizing sample nucleic acids and subsequent use in differential diagnosis of a disorder or disease. Accordingly, a sample nucleic acid sequence (including long-range sequence context) can be provided from analysis of a reference nucleic acid sequence with an error rate of less than 10%, 9%, 8%, 7%, 6%, 5%, 4%, 3%, 2%, 1%, 0.1%, 0.05%, 0.01%, 0.005%, 0.001%, 0.0005%, 0.0001%, 0.00005%, 0.00001%, or 0.000005%. In some cases, a sample nucleic acid sequence can be used for differential diagnosis of a disorder or disease (or absence thereof) by comparison with a sequence and/or characterization of a sequence of a reference nucleic acid with accuracy of at least 70%, 80%, 85%, 90%, 91%, 92%, 93%, 94%, 95%, 99%, 99.1%, 99.2%, 99.3% 99.4%, 99.5%, 99.6%, 99.7%, 99.8%, 99.9%, 99.95%, 99.99%, 99.995%, or 99.999%. In some cases, a sample nucleic acid sequence can be used for differential diagnosis of a disorder or disease (or absence thereof) by comparison with a sequence and/or characterization of a sequence of a reference nucleic acid with an error rate of less than 10%, 9%, 8%, 7%, 6%, 5%, 4%, 3%, 2%, 1%, 0.1%, 0.05%, 0.01%, 0.005%, 0.001%, 0.0005%, 0.0001%, 0.00005%, 0.00001%, or 0.000005%.

In an example, the methods and systems may be used to detect copy number variation in a patient with lung cancer in order to determine whether the lung cancer is Non-Small Cell Lung Cancer, which is associated with a variation in the EGFR gene. After such diagnosis, a patient's treatment regimen may be refined to correlate with the differential diagnosis. Targeted therapy or molecularly targeted therapy is one of the major modalities of medical treatment (pharmacotherapy) for cancer, others being hormonal therapy and cytotoxic chemotherapy. Targeted therapy blocks the growth of cancer cells by interfering with specific targeted molecules needed for carcinogenesis and tumor growth, rather than by simply interfering with all rapidly dividing cells (e.g. with traditional chemotherapy).

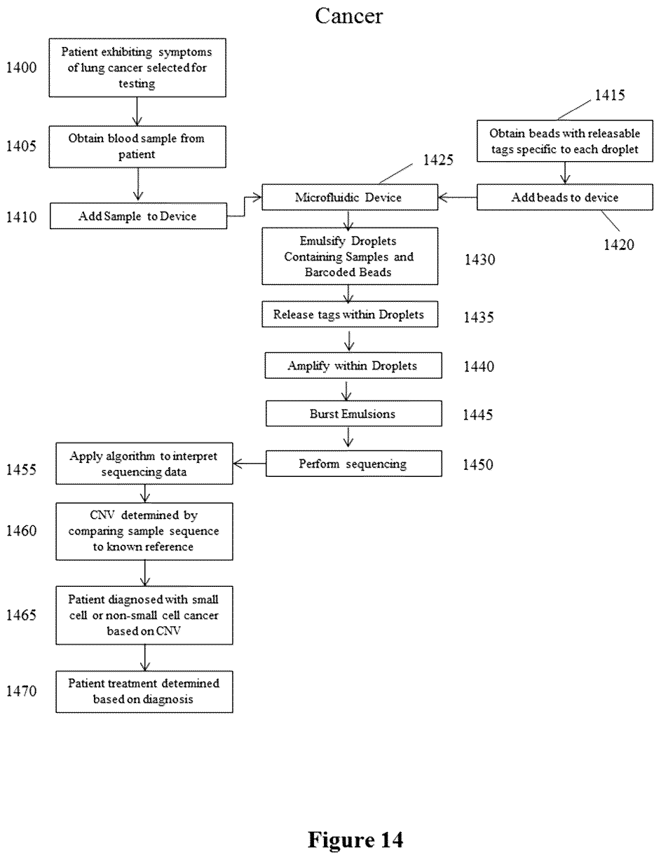

FIG. 14 shows an exemplary process for differentially diagnosing Non-Small Cell Lung Cancer. A patient with chromic cough, weight loss and shortness of breath is tested for lung cancer 1400. Blood is drawn from the patient 1405 and samples (e.g., circulating tumor cells, cell-free DNA, circulating nucleic acid (e.g., circulating tumor nucleic acid), etc.) are derived from the blood 1410. A set of barcoded beads may also be obtained, 1415. The beads can be linked to oligonucleotides containing one or more barcode sequences, as well as a primer, such as a random N-mer or other primer. In some cases, the barcode sequences are releasable from the barcoded beads, e.g., through cleavage of a linkage between the barcode and the bead or through degradation of the underlying bead to release the barcode, or a combination of the two. For example, in some aspects, the barcoded beads can be degraded or dissolved by an agent, such as a reducing agent to release the barcode sequences. In this example, a sample, 1410, barcoded beads, 1420, and, in some cases, other reagents, e.g., a reducing agent, are combined and subject to partitioning. By way of example, such partitioning may involve introducing the components to a droplet generation system, such as a microfluidic device, 1425. With the aid of the microfluidic device 1425, a water-in-oil emulsion 1430 may be formed, where the emulsion contains aqueous droplets that contain sample nucleic acid, 1410, barcoded beads, 1415, and, in some cases, a reducing agent. The reducing agent may dissolve or degrade the barcoded beads, thereby releasing the oligonucleotides with the barcodes and random N-mers from the beads within the droplets, 1435. The random N-mers may then prime different regions of the sample nucleic acid, resulting in amplified copies of the sample after amplification, where each copy is tagged with a barcode sequence, 1440. In some cases, each droplet contains a set of oligonucleotides that contain identical barcode sequences and different random N-mer sequences. Subsequently, the emulsion is broken, 1445 and additional sequences (e.g., sequences that aid in particular sequencing methods, additional barcodes, etc.) may be added, via, for example, amplification methods (e.g., PCR). Sequencing may then be performed, 1450, and an algorithm applied to interpret the sequencing data, 1455. Sequencing algorithms are generally capable, for example, of performing analysis of barcodes to align sequencing reads and/or identify the sample from which a particular sequence read belongs.

The analyzed sequence is then compared to a known genome reference sequence to determine the CNV of different genes 1460. If the EGFR copy number in the DNA is higher than normal, the patient can be differentially diagnosed with non-small cell lung cancer (NSCLC) instead of small-cell lung cancer 1465. The CTC of non-small cell lung cancer also has other copy number variations that may further distinguish it from small-cell lung cancer. Depending on the stage of the cancer, surgery, chemotherapy, or radiation therapy is prescribed 1470. In some cases, a patient diagnosed with NSLC is administered a drug targeted for such cancer such as an ALK inhibitor (e.g., Crizotinib). In some cases of variations in EGFR, the patient is administered cetuximab, panitumumab, lapatinib, and/or capecitabine. In a different situation, the target may be a different gene, such as ERBB2, and the therapy comprises trastuzumab (Herceptin). (2010) Nature466: 368-72; Cook E. H. and Scherer S. W. (2008) Nature 455: 919-923.

The main categories of targeted therapy are small molecules, small molecule drug conjugates and monoclonal antibodies. Small molecules may include tyrosine-kinase inhibitors such as Imatinib mesylate (Gleevec, also known as STI-571) (which is approved for chronic myelogenous leukemia, gastrointestinal stromal tumor and some other types of cancer); Gefitinib (Iressa, also known as ZD1839)(which targets the epidermal growth factor receptor (EGFR) tyrosine kinase and is approved in the U.S. for non small cell lung cancer); Erlotinib (marketed as Tarceva); Bortezomib (Velcade) (which is an apoptosis-inducing proteasome inhibitor drug that causes cancer cells to undergo cell death by interfering with proteins); tamoxifen; JAK inhibitors (e.g., tofactinib), ALK inhibitors (e.g., crizotinib.); Bcl-2 inhibitors (e.g. obatoclax in clinical trials, ABT-263, and Gossypol); PARP inhibitors (e.g. Iniparib, Olaparib in clinical trials); PI3K inhibitors (e.g. perifosine in a phase III trial). Apatinib (which is a selective VEGF Receptor 2 inhibitor); AN-152, (AEZS-108) doxorubicin linked to [D-Lys(6)]-LHRH; Braf inhibitors (vemurafenib, dabrafenib, LGX818) (used to treat metastatic melanoma that harbors BRAF V600E mutation); MEK inhibitors (trametinib, MEK162); CDK inhibitors, e.g. PD-0332991, LEE011 in clinical trials; Hsp90 inhibitors; and Salinomycin.

Other therapies include Small Molecule Drug Conjugates such as Vintafolide, which is a small molecule drug conjugate consisting of a small molecule targeting the folate receptor. Monoclonal antibodies are another type of therapy that may be administered as part of a method provided herein. Monoclonal drug conjugates may also be administered. Exemplary monoclonal antibodies include: Rituximab (marketed as MabThera or Rituxan)(which targets CD20 found on B cells and targets non Hodgkin lymphoma);Trastuzumab (Herceptin) (which targets the Her2/neu (also known as ErbB2) receptor expressed in some types of breast cancer); Cetuximab (marketed as Erbitux) and Panitumumab Bevacizumab (marketed as Avastin) (which targets VEGF ligand).

### VII. Characterizing Fetal Nucleic Acid From Parental Nucleic Acid

As noted elsewhere herein, the methods and systems described herein may also be used to characterize circulating nucleic acids within the blood or plasma of a subject. Such analyses include the analysis of circulating tumor DNA, for use in identification of potential disease states in a patient, or circulating fetal DNA within the blood or plasma of a pregnant female, in order to characterize the fetal DNA in a non-invasive way, e.g., without resorting to direct sampling through amniocentesis or other invasive procedures.

In some cases, the methods may be used to characterize fetal nucleic acid sequences, e.g. circulating fetal DNA, based, at least in part, on analysis of parental nucleic acid sequences. For example, long range sequence context can be determined for both paternal and maternal nucleic acids (e.g., having lengths of longer than 1 kb, longer than 5 kb, longer than 10 kb, longer than 15 kb, longer than 20 kb, longer than 30 kb, longer than 40 kb, longer than 50 kb, longer than 60 kb, longer than 70 kb, longer than 80 kb, longer than 90 kb or even longer than 100 kb) from shorter barcoded fragments using methods and systems described herein. Long range sequence context can be used to determine one or more haplotypes and one or more genetic variations, including single nucleotide polymorphisms (SNPs), structural variations in (e.g., a copy number variation, an insertion, a deletion, a translocation, an inversion, a rearrangement, a repeat expansion, a retrotransposon, a duplication, a gene fusion, etc.) in both the paternal and maternal nucleic acid sequences. Moreover, long range sequence context of paternal and maternal nucleic acids and any determined SNP, haplotype and/or structural variation information can be used to characterize a sequence of a fetal nucleic acid obtained from the pregnant mother (e.g., circulating fetal nucleic acid, such as, for example, cell-free fetal nucleic acid). In some cases, characterizations of a fetal nucleic acid, via comparison with maternal and paternal sequences and characterization, may be completed with the aid of a programmed computer processor. In some cases, such a programmed computer processor can be included in a computer control system, such as in an example computer control system described elsewhere herein.

For example, a sequence and/or long range sequence context of parental and/or maternal nucleic acids may be used as a reference by which to characterize fetal nucleic acid, including a fetal nucleic acid sequence. Indeed, long range sequence context obtained by methods and systems described herein can provide improved, long range sequence context information for paternal and maternal nucleic acids from which fetal nucleic acid sequences can be characterized. In some cases, characterization of a fetal nucleic acid sequence from parental nucleic acids as references may include determining a sequence for at least a portion of a fetal nucleic acid, and/or calling one or more SNPs of a fetal nucleic acid sequence, determining one or more de novo mutations of a fetal nucleic acid sequence, determining one or more haplotypes of a fetal nucleic acid sequence, and/or determining and characterizing one or more structural variations, etc. in a sequence of the fetal nucleic acid.

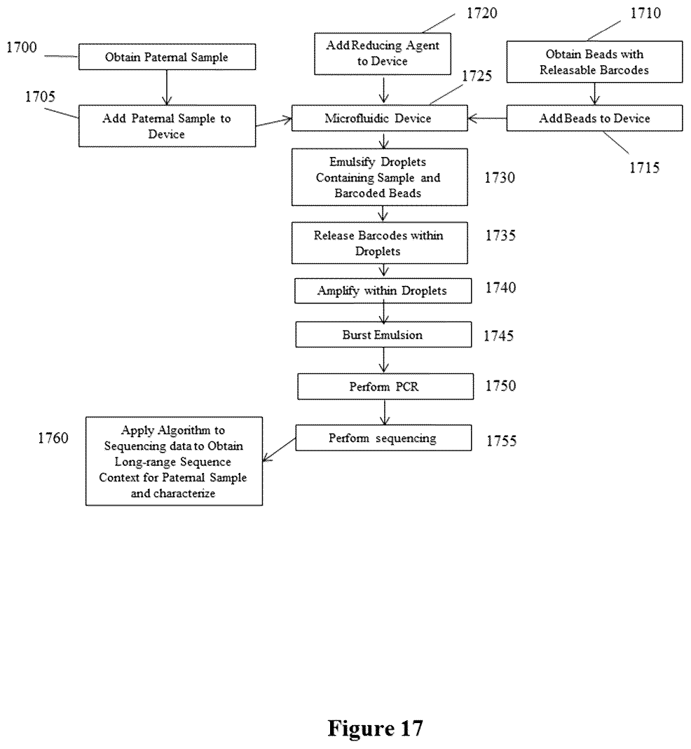

FIGS. 17-19 illustrate an example method for characterizing fetal nucleic acid from longer range sequence context obtained for paternal and maternal nucleic acid, via sequencing of shorter barcoded fragments. FIG. 17 demonstrates an example method by which longer range sequence context can be determined for a paternal nucleic acid sample (e.g., paternal genomic DNA) from shorter barcoded fragments, such as, for example, in a manner analogous to that shown in FIG. 6. With respect to FIG. 17, a sample comprising paternal nucleic acid may be obtained from the father of a fetus, 1700, and a set of barcoded beads may also be obtained, 1710. The beads can be linked to oligonucleotides containing one or more barcode sequences, as well as a primer, such as a random N-mer or other primer. In some cases, the barcode sequences are releasable from the barcoded beads, e.g., through cleavage of a linkage between the barcode and the bead or through degradation of the underlying bead to release the barcode, or a combination of the two. For example, in some aspects, the barcoded beads can be degraded or dissolved by an agent, such as a reducing agent to release the barcode sequences. In this example, paternal sample comprising nucleic acid, 1705, barcoded beads, 1715, and, in some cases, other reagents, e.g., a reducing agent, 1720, are combined and subject to partitioning. In some cases, the paternal sample 1700 is fragmented prior to partitioning and at least some of the resulting fragments are partitioned as 1705 for barcoding. By way of example, such partitioning may involve introducing the components to a droplet generation system, such as a microfluidic device, 1725. With the aid of the microfluidic device 1725, a water-in-oil emulsion 1730 may be formed, where the emulsion contains aqueous droplets that contain paternal sample nucleic acid, 1705, reducing agent, 1720, and barcoded beads, 1715. The reducing agent may dissolve or degrade the barcoded beads, thereby releasing the oligonucleotides with the barcodes and random N-mers from the beads within the droplets, 1735. The random N-mers may then prime different regions of the paternal sample nucleic acid, resulting in amplified copies of the paternal sample after amplification, where each copy is tagged with a barcode sequence, 1740. In some cases, amplification 1740 may be achieved by a method analogous to that described elsewhere herein and schematically depicted in FIG. 5. In some cases, each droplet contains a set of oligonucleotides that contain identical barcode sequences and different random N-mer sequences. Subsequently, the emulsion is broken, 1745 and additional sequences (e.g., sequences that aid in particular sequencing methods, additional barcodes, etc.) may be added, via, for example, amplification methods, 1750 (e.g., PCR). Sequencing may then be performed, 1755, and an algorithm applied to interpret the sequencing data 1760. In some cases, for example, interpretation of sequencing data 1760 may include providing a sequence for at least a portion of the paternal nucleic acid. In some cases, long range sequence context for the paternal nucleic acid sample can be obtained and characterized (e.g., determination of one or more haplotypes as described elsewhere herein, determination of one or more structural variations (e.g., a copy number variation, an insertion, a deletion, a translocation, an inversion, a rearrangement, a repeat expansion, a duplication, a retrotransposon, a gene fusion, etc.), calling of one or more SNPs, determination of one or more other genetic variations, etc.). In some cases, variants can be called for various paternal nucleic acids and inferred contigs generated to provide longer range sequence context, such as is described elsewhere herein with respect to FIG. 7.

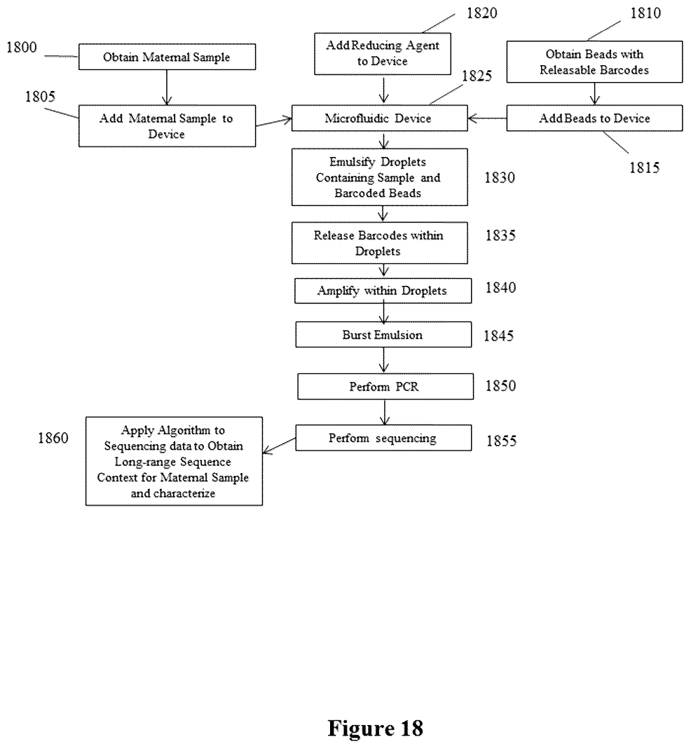

FIG. 18 demonstrates an example method by which long range sequence context can be determined for a maternal nucleic acid sample (e.g., maternal genomic DNA) from shorter barcoded fragments, such as, for example, in a manner analogous to that shown in FIG. 6. With respect to FIG. 18, a sample comprising maternal nucleic acid may be obtained from the pregnant mother of a fetus, 1800, and a set of barcoded beads may also be obtained, 1810. The beads can be linked to oligonucleotides containing one or more barcode sequences, as well as a primer, such as a random N-mer or other primer. In some cases, the barcode sequences are releasable from the barcoded beads, e.g., through cleavage of a linkage between the barcode and the bead or through degradation of the underlying bead to release the barcode, or a combination of the two. For example, in some aspects, the barcoded beads can be degraded or dissolved by an agent, such as a reducing agent to release the barcode sequences. In this example, maternal sample comprising nucleic acid, 1805, barcoded beads, 1815, and, in some cases, other reagents, e.g., a reducing agent, 1820, are combined and subject to partitioning. In some cases, the maternal sample 1800 is fragmented prior to partitioning and at least some of the resulting fragments are partitioned as 1805 for barcoding. By way of example, such partitioning may involve introducing the components to a droplet generation system, such as a microfluidic device, 1825. With the aid of the microfluidic device 1825, a water-in-oil emulsion 1830 may be formed, where the emulsion contains aqueous droplets that contain maternal sample nucleic acid, 1805, reducing agent, 1820, and barcoded beads, 1815. The reducing agent may dissolve or degrade the barcoded beads, thereby releasing the oligonucleotides with the barcodes and random N-mers from the beads within the droplets, 1835. The random N-mers may then prime different regions of the maternal sample nucleic acid, resulting in amplified copies of the maternal sample after amplification, where each copy is tagged with a barcode sequence, 1840. In some cases, amplification 1840 may be achieved by a method analogous to that described elsewhere herein and schematically depicted in FIG. 5. In some cases, each droplet contains a set of oligonucleotides that contain identical barcode sequences and different random N-mer sequences. Subsequently, the emulsion is broken, 1845 and additional sequences (e.g., sequences that aid in particular sequencing methods, additional barcodes, etc.) may be added, via, for example, amplification methods, 1850 (e.g., PCR). Sequencing may then be performed, 1855, and an algorithm applied to interpret the sequencing data, 1860. In some cases, for example, interpretation of sequencing data 1860 may include providing a sequence for at least a portion of the maternal nucleic acid. In some cases, long range sequence context for the maternal nucleic acid sample can be obtained and characterized (e.g., determination of one or more haplotypes as described elsewhere herein, determination of one or more structural variations (e.g., a copy number variation, an insertion, a deletion, a translocation, an inversion, a rearrangement, a repeat expansion, a duplication, a retrotransposon, a gene fusion, etc.), calling of one or more SNPs, determination of one or more other genetic variations, etc. In some cases, variants can be called for various maternal nucleic acids obtained from a sample and inferred contigs generated to provide longer range sequence context, such as is described elsewhere herein with respect to FIG. 7.

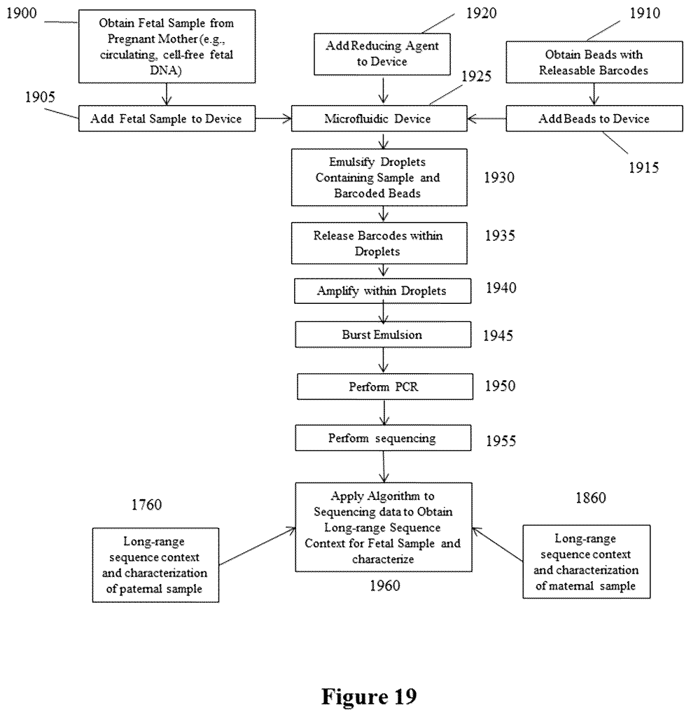

FIG. 19 demonstrates an example of characterizing a fetal sample sequence from the paternal 1760 and maternal 1860 characterizations obtained as shown in FIG. 17 and FIG. 18, respectively. As shown in FIG. 19, a fetal nucleic acid sample can be obtained from the pregnant mother 1900. Long range sequence context can be obtained for the fetal nucleic acid from sequencing of shorter barcoded fragments as is described elsewhere herein, such as, for example, via the method schematically depicted in FIG. 6. In some cases, the fetal nucleic acid sample may be circulating fetal DNA and/or cell-free DNA that may be, for example, obtained from the pregnant mother's blood, plasma, other bodily fluid, or tissue. A set of barcoded beads may also be obtained, 1910. The beads are can be linked to oligonucleotides containing one or more barcode sequences, as well as a primer, such as a random N-mer or other primer. In some cases, the barcode sequences are releasable from the barcoded beads, e.g., through cleavage of a linkage between the barcode and the bead or through degradation of the underlying bead to release the barcode, or a combination of the two. For example, in some aspects, the barcoded beads can be degraded or dissolved by an agent, such as a reducing agent to release the barcode sequences. In this example, fetal sample comprising nucleic acid, 1905, barcoded beads, 1915, and, in some cases, other reagents, e.g., a reducing agent, 1920, are combined and subject to partitioning as 1905. In some cases, the fetal sample 1900 is fragmented prior to partitioning and at least some of the resulting fragments are partitioned as 1905 for barcoding. By way of example, such partitioning may involve introducing the components to a droplet generation system, such as a microfluidic device, 1925. With the aid of the microfluidic device 1925, a water-in-oil emulsion 1930 may be formed, where the emulsion contains aqueous droplets that contain maternal sample nucleic acid, 1905, reducing agent, 1920, and barcoded beads, 1915. The reducing agent may dissolve or degrade the barcoded beads, thereby releasing the oligonucleotides with the barcodes and random N-mers from the beads within the droplets, 1935. The random N-mers may then prime different regions of the fetal sample nucleic acid, resulting in amplified copies of the fetal sample after amplification, where each copy is tagged with a barcode sequence, 1940. In some cases, amplification 1940 may be achieved by a method analogous to that described elsewhere herein and schematically depicted in FIG. 5. In some cases, each droplet contains a set of oligonucleotides that contain identical barcode sequences and different random N-mer sequences. Subsequently, the emulsion is broken, 1945 and additional sequences (e.g., sequences that aid in particular sequencing methods, additional barcodes, etc.) may be added, via, for example, amplification methods, 1950 (e.g., PCR). Sequencing may then be performed, 1955, and an algorithm applied to interpret the sequencing data, 1960. In general, longer range sequence context for the fetal nucleic acid sample can be obtained from the shorter barcoded fragments that are sequenced. In some cases, for example, interpretation of sequencing data 1960 may include providing a sequence for at least a portion of the fetal nucleic acid. The fetal nucleic acid sequence can be characterized 1960 (e.g., determination of one or more haplotypes as described elsewhere herein, determination of one or more structural variations (e.g., a copy number variation, an insertion, a deletion, a translocation, an inversion, a rearrangement, a repeat expansion, a duplication, retrotransposon, a gene fusion, etc.), determination of one or more de novo mutations, calling of one or more SNPs, etc.) using the long-range sequence contexts and/or characterizations of the paternal 1760 and maternal 1860 samples. In some cases, phase blocks of the fetal nucleic acid can be determined by comparison of the fetal nucleic acid sequence to the maternal and paternal phase blocks.

As can be appreciated, analysis of paternal nucleic acid, maternal nucleic acid and/or fetal nucleic acid may completed as part of separate partitioning analyses or may be completed as part of one or more combined partitioning analyses. For example, paternal, maternal and fetal nucleic acids may be added to the same device and barcoded maternal, paternal and fetal fragments generated in droplets according to FIGS. 17-19, where an emulsion comprises the droplets for the three types of nucleic acid. The emulsion can then be broken and the contents of the droplets pooled, further processed (e.g., bulk addition of additional sequences via PCR) and sequenced as described elsewhere herein. Individual sequencing reads from the barcoded fragments can be attributed to their respective sample sequence via barcode sequences.

In some cases, the sequence of a fetal nucleic acid, including the sequence of the fetal genome, and/or genetic variations in the fetal nucleic acid sequence may be determined from long range paternal and maternal sequence contexts and characterizations obtained using methods and systems described herein. For example, genome sequencing of paternal and maternal genomes, along with sequencing of circulating fetal nucleic acids, may be used to determine a corresponding fetal genome sequence. An example of determining a sequence of genomic fetal nucleic acid from sequence analysis of parental genomes and cell-free fetal nucleic acid can be found in Kitzman et al. (2012 Jun. 6) Sci Transl. Med. 4(137): 137ra76, which is herein entirely incorporated by reference. Determination of a fetal genome may be useful in the prenatal determination and diagnosis of genetic disorders in the fetus, including, for example, fetal aneuploidy. As discussed elsewhere herein, methods and systems provided herein can be useful in resolving haplotypes in nucleic acid sequences. Haplotype-resolved paternal and maternal sequences can be determined for paternal and maternal sample nucleic acid sequences, respectively which can aid in more accurately determining the sequence of a fetal genome and/or characterizing the same.

Utilizing methods and systems herein can improve accuracy in determining long range sequence context of nucleic acids, including the long-range sequence context of parental nucleic acid sequences (e.g., maternal nucleic acid sequences, paternal nucleic acid sequences). The methods and systems provided herein may determine long-range sequence context of parental nucleic acids with accuracy of at least 70%, 80%, 85%, 90%, 91%, 92%, 93%, 94%, 95%, 99%, 99.1%, 99.2%, 99.3% 99.4%, 99.5%, 99.6%, 99.7%, 99.8%, 99.9%, 99.95%, 99.99%, 99.995%, or 99.999%. In some cases, the methods and systems provided herein may determine long-range sequence context of parental nucleic acids with an error rate of less than 10%, 9%, 8%, 7%, 6%, 5%, 4%, 3%, 2%, 1%, 0.1%, 0.05%, 0.01%, 0.005%, 0.001%, 0.0005%, 0.0001%, 0.00005%, 0.00001%, or 0.000005%. Moreover, methods and systems herein can also improve accuracy in characterizing a paternal nucleic acid sequence in one or more aspects (e.g., determination of a sequence, determination of one or more genetic variations, determination of one or more structural variants, determination of haplotypes, etc.). Accordingly, the methods and systems provided herein may characterize a paternal nucleic acid sequence in one or more aspects with an accuracy of at least 70%, 80%, 85%, 90%, 91%, 92%, 93%, 94%, 95%, 99%, 99.1%, 99.2%, 99.3% 99.4%, 99.5%, 99.6%, 99.7%, 99.8%, 99.9%, 99.95%, 990.99%, 990.995%, or 990.999%. In some cases, the methods and systems provided herein may characterize a parental nucleic acid sequence in one or more aspects with an error rate of less than 10%, 9%, 8%, 7%, 6%, 5%, 4%, 3%, 2%, 1%, 0.1%, 0.05%, 0.01%, 0.005%, 0.001%, 0.0005%, 0.0001%, 0.00005%, 0.00001%, or 0.000005%.

Moreover, as is discussed above, improved accuracy in determining long-range sequence context of parental nucleic acids and characterization of the same can result in improved accuracy in sequencing and characterizing fetal nucleic acids. Accordingly, in some cases, a fetal nucleic acid sequence (including long-range sequence context) can be provided from analysis of parental nucleic sequences with accuracy of at least 70%, 80%, 85%, 90%, 91%, 92%, 93%, 94%, 95%, 99%, 99.1%, 99.2%, 99.3% 99.4%, 99.5%, 99.6%, 99.7%, 99.8%, 99.9%, 99.95%, 99.99%, 99.995%, or 99.999%. In some cases, a fetal nucleic acid sequence (including long-range sequence context) can be provided from analysis of parental nucleic sequences with an error rate of less than 10%, 9%, 8%, 7%, 6%, 5%, 4%, 3%, 2%, 1%, 0.1%, 0.05%, 0.01%, 0.005%, 0.001%, 0.0005%, 0.0001%, 0.00005%, 0.00001%, or 0.000005%. In some cases, a fetal nucleic acid sequence can be characterized in one or more aspects via analysis of parental nucleic acid sequences as described herein (e.g., determination of a sequence, determination of one or more genetic variations, determination of one or more structural variations, determination of haplotypes, etc.) with accuracy of at least 70%, 80%, 85%, 90%, 91%, 92%, 93%, 94%, 95%, 99%, 99.1%, 99.2%, 99.3% 99.4%, 99.5%, 99.6%, 99.7%, 99.8%, 99.9%, 99.95%, 99.99%, 99.995%, or 99.999%. In some cases, a fetal nucleic acid sequence can be characterized in one or more aspects via analysis of parental nucleic acid sequences as described herein (e.g., determination of a sequence, determination of one or more genetic variations, determination of haplotypes, determination of one or more structural variations, etc.) with an error rate of less than 10%, 9%, 8%, 7%, 6%, 5%, 4%, 3%, 2%, 1%, 0.1%, 0.05%, 0.01%, 0.005%, 0.001%, 0.0005%, 0.0001%, 0.00005%, 0.00001%, or 0.000005%. VIII.

### Samples

Detection of a disease or disorder may begin with obtaining a sample from a patient. The term “sample,” as used herein, generally refers to a biological sample. Examples of biological samples include nucleic acid molecules, amino acids, polypeptides, proteins, carbohydrates, fats, or viruses. In an example, a biological sample is a nucleic acid sample including one or more nucleic acid molecules. Exemplary samples may include polynucleotides, nucleic acids, oligonucleotides, cell-free nucleic acid (e.g., cell-free DNA (cfDNA)), circulating cell-free nucleic acid, circulating tumor nucleic acid (e.g., circulating tumor DNA (ctDNA)), circulating tumor cell (CTC) nucleic acids, nucleic acid fragments, nucleotides, DNA, RNA, peptide polynucleotides, complementary DNA (cDNA), double stranded DNA (dsDNA), single stranded DNA (ssDNA), plasmid DNA, cosmid DNA, chromosomal DNA, genomic DNA (gDNA), viral DNA, bacterial DNA, mtDNA (mitochondrial DNA), ribosomal RNA, cell-free DNA, cell free fetal DNA (cffDNA), mRNA, rRNA, tRNA, nRNA, siRNA, snRNA, snoRNA, scaRNA, microRNA, dsRNA, viral RNA, and the like. In summary, the samples that are used may vary depending on the particular processing needs.

Any substance that comprises nucleic acid may be the source of a sample. The substance may be a fluid, e.g., a biological fluid. A fluidic substance may include, but not limited to, blood, cord blood, saliva, urine, sweat, serum, semen, vaginal fluid, gastric and digestive fluid, spinal fluid, placental fluid, cavity fluid, ocular fluid, serum, breast milk, lymphatic fluid, or combinations thereof. The substance may be solid, for example, a biological tissue. The substance may comprise normal healthy tissues, diseased tissues, or a mix of healthy and diseased tissues. In some cases, the substance may comprise tumors. Tumors may be benign (non-cancer) or malignant (cancer). Non-limiting examples of tumors may include: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's, leiomyosarcoma, rhabdomyosarcoma, gastrointestinal system carcinomas, colon carcinoma, pancreatic cancer, breast cancer, genitourinary system carcinomas, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, endocrine system carcinomas, testicular tumor, lung carcinoma, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, or combinations thereof. The substance may be associated with various types of organs. Non-limiting examples of organs may include brain, liver, lung, kidney, prostate, ovary, spleen, lymph node (including tonsil), thyroid, pancreas, heart, skeletal muscle, intestine, larynx, esophagus, stomach, or combinations thereof. In some cases, the substance may comprise a variety of cells, including but not limited to: eukaryotic cells, prokaryotic cells, fungi cells, heart cells, lung cells, kidney cells, liver cells, pancreas cells, reproductive cells, stem cells, induced pluripotent stem cells, gastrointestinal cells, blood cells, cancer cells, bacterial cells, bacterial cells isolated from a human microbiome sample, etc. In some cases, the substance may comprise contents of a cell, such as, for example, the contents of a single cell or the contents of multiple cells. Methods and systems for analyzing individual cells are provided in, e.g., U.S. Provisional Patent Application No. 62/017,558, filed Jun. 26, 2014, the full disclosure of which is hereby incorporated by reference in its entirety.

Samples may be obtained from various subjects. A subject may be a living subject or a dead subject. Examples of subjects may include, but not limited to, humans, mammals, non-human mammals, rodents, amphibians, reptiles, canines, felines, bovines, equines, goats, ovines, hens, avines, mice, rabbits, insects, slugs, microbes, bacteria, parasites, or fish. In some cases, the subject may be a patient who is having, suspected of having, or at a risk of developing a disease or disorder. In some cases, the subject may be a pregnant woman. In some case, the subject may be a normal healthy pregnant woman. In some cases, the subject may be a pregnant woman who is at a risking of carrying a baby with certain birth defect.

A sample may be obtained from a subject by various approaches. For example, a sample may be obtained from a subject through accessing the circulatory system (e.g., intravenously or intra-arterially via a syringe or other apparatus), collecting a secreted biological sample (e.g., saliva, sputum urine, feces, etc.), surgically (e.g., biopsy) acquiring a biological sample (e.g., intra-operative samples, post-surgical samples, etc.), swabbing (e.g., buccal swab, oropharyngeal swab), or pipetting.

CNVs can be associated with efficacy of a therapy. For example, increased HER2 gene copy number can enhance the response to gefitinib therapy in advanced non-small cell lung cancer. See Cappuzzo F. et al. (2005) J. Clin. Oncol. 23: 5007-5018. High EGFR gene copy number can predict for increased sensitivity to lapatinib and capecitabine. See Fabi et al. (2010) J. Clin. Oncol. 28:15s (2010 ASCO Annual Meeting). High EGFR gene copy number is associated with increased sensitivity to cetuximab and panitumumab.

Copy number variations can be associated with resistance of cancer patients to certain therapeutics. For example, amplification of thymidylate synthase can result in resistance to 5-fluorouracil treatment in metastatic colorectal cancer patients. See Wang et al. (2002) PNAS USA vol. 99, pp. 16156-61.

IX. Computer Control Systems

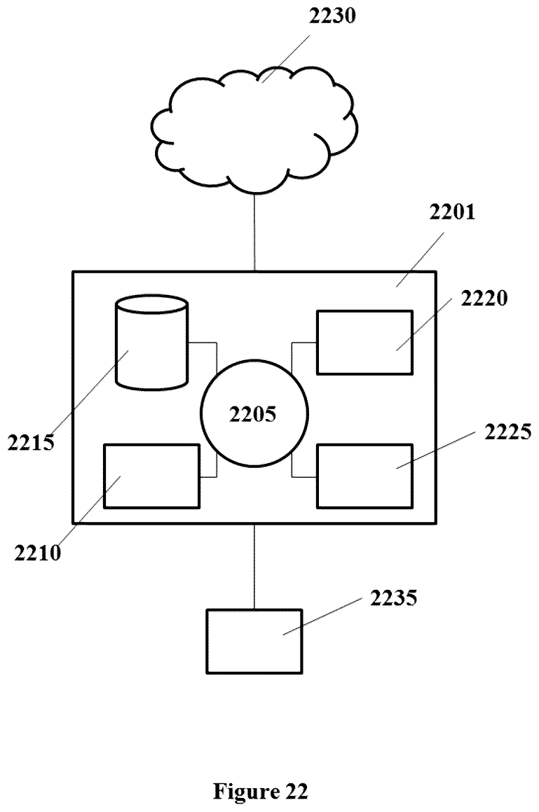

The present disclosure provides computer systems that are programmed or otherwise configured to implement methods provided herein, such as, for example, methods for nucleic sequencing and determination of genetic variations, storing reference nucleic acid sequences, conducting sequence analysis and/or comparing sample and reference nucleic acid sequences as described herein. An example of such a computer system is shown in FIG. 22. As shown in FIG. 22, the computer system 2201 includes a central processing unit (CPU, also “processor” and “computer processor” herein) 2205, which can be a single core or multi core processor, or a plurality of processors for parallel processing. The computer system 2201 also includes memory or memory location 2210 (e.g., random-access memory, read-only memory, flash memory), electronic storage unit 2215 (e.g., hard disk), communication interface 2220 (e.g., network adapter) for communicating with one or more other systems, and peripheral devices 2225, such as cache, other memory, data storage and/or electronic display adapters. The memory 2210, storage unit 2215, interface 2220 and peripheral devices 2225 are in communication with the CPU 2205 through a communication bus (solid lines), such as a motherboard. The storage unit 2215 can be a data storage unit (or data repository) for storing data. The computer system 2201 can be operatively coupled to a computer network (“network”) 2230 with the aid of the communication interface 2220. The network 2230 can be the Internet, an internet and/or extranet, or an intranet and/or extranet that is in communication with the Internet. The network 2230 in some cases is a telecommunication and/or data network. The network 2230 can include one or more computer servers, which can enable distributed computing, such as cloud computing. The network 2230, in some cases with the aid of the computer system 2201, can implement a peer-to-peer network, which may enable devices coupled to the computer system 2201 to behave as a client or a server.

The CPU 2205 can execute a sequence of machine-readable instructions, which can be embodied in a program or software. The instructions may be stored in a memory location, such as the memory 2210. Examples of operations performed by the CPU 2205 can include fetch, decode, execute, and writeback.

The storage unit 2215 can store files, such as drivers, libraries and saved programs. The storage unit 2215 can store user data, e.g., user preferences and user programs. The computer system 2201 in some cases can include one or more additional data storage units that are external to the computer system 2201, such as located on a remote server that is in communication with the computer system 2201 through an intranet or the Internet.

The computer system 2201 can communicate with one or more remote computer systems through the network 2230. For instance, the computer system 2201 can communicate with a remote computer system of a user (e.g., operator). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 2201 via the network 2230.

Methods as described herein can be implemented by way of machine (e.g., computer processor) executable code stored on an electronic storage location of the computer system 2201, such as, for example, on the memory 2210 or electronic storage unit 2215. The machine executable or machine readable code can be provided in the form of software. During use, the code can be executed by the processor 2205. In some cases, the code can be retrieved from the storage unit 2215 and stored on the memory 2210 for ready access by the processor 2205. In some situations, the electronic storage unit 2215 can be precluded, and machine-executable instructions are stored on memory 2210.

The code can be pre-compiled and configured for use with a machine have a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 2201, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 2201 can include or be in communication with an electronic display 2235 that comprises a user interface (UI) for providing, for example, an output or readout of a nucleic acid sequencing instrument coupled to the computer system 2201. Such readout can include a nucleic acid sequencing readout, such as a sequence of nucleic acid bases that comprise a given nucleic acid sample. The UI may also be used to display the results of an analysis making use of such readout. Examples of UI's include, without limitation, a graphical user interface (GUI) and web-based user interface. The electronic display 2235 can be a computer monitor, or a capacitive or resistive touchscreen.

### EXAMPLES

### Example 1: Identification of Phased Variants

Genomic DNA from the NA12878 human cell line was subjected to size based separation of fragments using a Blue Pippin DNA sizing system to recover fragments that were approximately 10 kb in length. The size selected sample nucleic acids were then copartitioned with barcode beads in aqueous droplets within a fluorinated oil continuous phase using a microfluidic partitioning system (see e.g., U.S. Provisional Patent Application No. 61/977,804, filed Apr. 10, 2014, and incorporated herein by reference in its entirety for all purposes), where the aqueous droplets also included the dNTPs, thermostable DNA polymerase and other reagents for carrying out amplification within the droplets, as well as a chemical activator for releasing the barcode oligonucleotides from the beads. This was repeated both for 1 ng of total input DNA and 2 ng of total input DNA. The barcode beads were obtained as a subset of a stock library that represented barcode diversity of over 700,000 different barcode sequences. The barcode containing oligonucleotides included additional sequence components and had the general structure:

Bead-P5-BC-R1-Nmer

Where P5 and R1 refer to the Illumina attachment and Read1 primer sequences, respectively, BC denotes the barcode portion of the oligonucleotide, and N-mer denotes a random 10 base N-mer priming sequence used to prime the template nucleic acids. See, e.g., U.S. patent application Ser. No. 14/316,383, filed Jun. 26, 2014, the full disclosure of which is hereby incorporated herein by reference in its entirety for all purposes.

Following bead dissolution, the droplets were thermocycled to allow for primer extension of the barcode oligos against the template of the sample nucleic acids within each droplet. This resulted in copied fragments of the sample nucleic acids that included the barcode sequence representative of the originating partition, in addition to the other included sequences set forth above.

After barcode labeling of the copy fragments, the emulsion of droplets including the amplified copy fragments was broken and the additional sequencer required components, e.g., read2 primer sequence and P7 attachment sequence for Illumina sequencer, were added to the copy fragments through additional amplification, which attached these sequences to the other end of the copy fragments.

The sequencing library was then sequenced on an Illumina HiSeq system at 10× coverage, 20× coverage and 30× coverage, and the resulting sequence reads and their associated barcode sequences were then analyzed. Proximally mapping sequences that shared common barcodes were then assembled into larger contigs, and single nucleotide polymorphisms were identified and associated with individual starting molecules based upon their associated barcodes and sequence mapping, to identify phased SNPs. Sequences that included overlapping phased SNPs were then assembled into phase blocks or inferred contigs of phased sequence data based upon the overlapping phased SNPs. The resulting data was compared to known haplotype maps for the cell line for comparison.

In at least one approach, each allele of a series of heterozygous variants is assigned to one of two to two haplotypes. A log-likelihood function log P(barcoded reads | phasing assignment, variants) is defined that returns the log-likelihood of the observed read and barcode data, given a set of variants, and a phasing assignment of the heterozygous variants. The form of the log-likelihood function derives from two main observations about barcoded sequence read data: (1) The reads from one barcode cover a small fraction of a haploid genome, so the probability of one barcode containing reads for both haplotypes in a given region of the genome is small. Conversely, the reads for one barcode in a local region of the genome are very likely to come from a single haplotype; (2) the probability that an observed base differs from the true base in haplotype it was derived from is described by the Phred QV of the observed base assigned by the sequencer.

The phasing configuration that maximizes the log-likelihood function, for a given set of barcoded reads and variants is then reported. The maximum-likelihood scoring haplotype configuration is then found by a structured search procedure. First, a beam search is used to find an optimal phasing configuration of a small block of neighboring variants (e.g., ˜50 variants). Second the relative phasing of the blocks is determined in a sweep over the block junctions. At this point an overall near-optimal phasing configuration is found and is used as a starting point for further optimization. The haplotype assignment of individual variants is then inverted to find local improvement to the phasing, the difference in the log-likelihood between the swapped configurations provides an estimate of the confidence of that phasing assignment. Finally the phasing configuration is broken into phase blocks that have a high probability of being internally correct. It is then tested whether to break a phase block at each SNP by comparing the log-likelihoods of the optimal configuration with a configuration where all SNPs right of the current SNP have their haplotype assignment inverted.

The table below, provides the phasing metrics obtained for the NA 12878 genome. As is apparent, extremely long phase blocks are obtained from short read sequence data, correctly identifying significant percentages of phased SNPs, with very low short or long switch errors.

| | 10X | 20X | 30X | 30X Beam |
|---|---|---|---|---|
| | Coverage | Coverage | Coverage | Search |
| N50 Phase Block | 193 kb | 385 kb | 428 kb | 489 kb |
| Longest Phase Block | 2121 kb | 2514 kb | 2514 kb | 3027 kb |
| Long Switch Error | 0.0053 | 0.0021 | 0.0018 | 0.0015 |
| Short Switch Error | 0.004 | 0.0017 | 0.0014 | 0.0012 |
| SNPs Phased | 83% | 94% | 95% | 95.2% |

Further experiments phased SNPs from a number of additional samples including the NA12878 trio (NA12878, NA12882 and NA12877), Gujarati (NA20847), Mexican (NA19662) and African (NA19701) cell line samples. N50 phase block lengths of approximately 1 MB were achieved with greater than 95% of the SNPs phased with switch errors of less than 0.3%. Whole exome sequencing of the same samples, e.g., where targeted pull down followed the barcoding, showed genic SNP phasing of approximately 90% again with switch errors of less than 0.3%.

### Example 2: Identification of EML-4/ALK Gene Inversions/Translocations

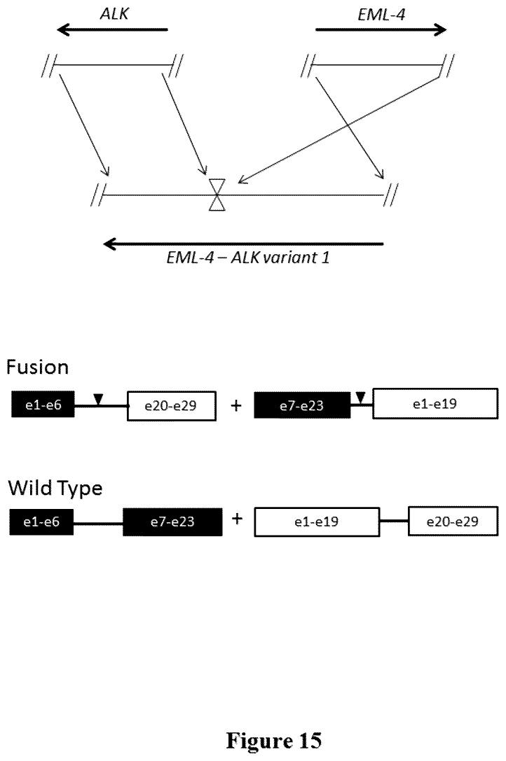

The methods and processes described herein were used to detect structural variations from a characterized cancer cell line. In particular, NCI-H2228 lung cancer cell line is known to have an EML4-ALK fusion translocation within its genome. The structure of the variation compared to wild type is illustrated in FIG. 15. As shown in the top panel, in the variant structure, the EML-4 gene, while on the same chromosome, is relatively separate or distant from the ALK gene, is instead translocated and fused to the ALK gene (See e.g., Choi, et al., Identification of Novel Isoforms of the EML4-LK Transforming Gene in Non-Small Cell Lung Cancer, J. Cancer Res., 68:4971 (July 2008)). In conjunction with the translocation, the EML4 gene is also inverted. The translocation is further illustrated in Panel II, as compared to the wild type structure, where the translocation results in the fusion of exons 1-6 of EML-4 (shown as black boxes) to exons 20-29 of ALK (shown as white boxes), as well as the fusion of exons 7-23 of ALK fused to exons 1-19 of the EML-4.

In order to identify this variation, genomic DNA from the NCI-H2228 cell line was subjected to size separation using a Blue Pippin® system (Sage Sciences, Inc.), to select for fragments of approximately 10 kb in length.

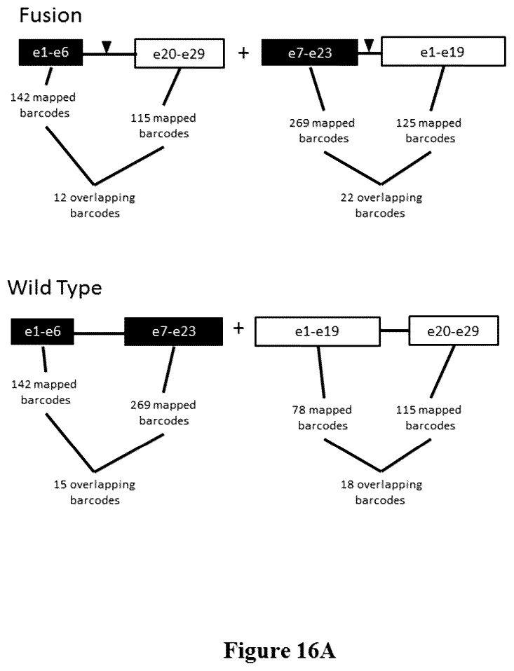

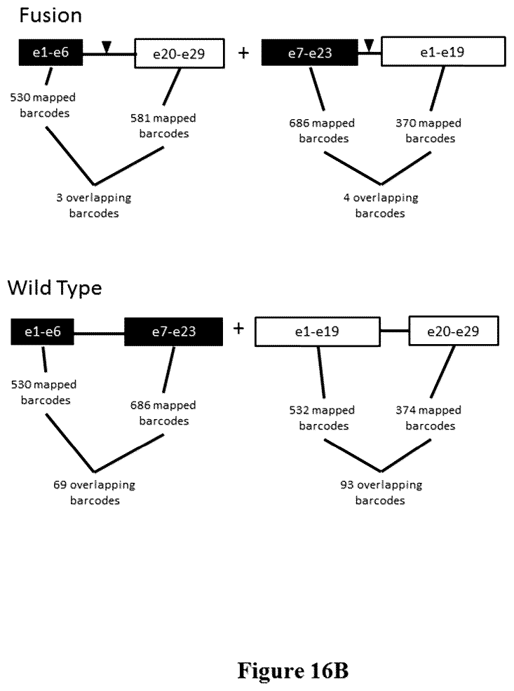

The size selected sample nucleic acids were then copartitioned with barcode beads, amplified and processed into a sequencing library as described above for Example 1, except that the DNA was subjected to hybrid capture using an Agilent SureSelect Exome capture kit after barcoding and prior to sequencing. The sequencing library was then sequenced to approximately 80× coverage on an Illumina HiSeq system and the resulting sequence reads and their associated barcode sequences were then analyzed. The higher number of shared barcodes among portions of the genome that span the translocation event was clearly evident as compared to the wild type, illustrating structural proximity between the fused components where not present in the wild type. In particular, and as shown in FIG. 16A, the fusion structure showed barcode overlap between EML-4 exons 1-6 and ALK exons 20-29, of 12 barcodes, and between EML-4 exons 7-23 and ALK exons 1-19, of 20 barcodes, that were comparable to the overlapping barcodes for the wild type construct for the heterozygous cell line.

In contrast, a negative control run using a non variant cell line (NA12878) showed substantially only barcode overlap for the wild type vs. the variant construct, as shown in FIG. 16B, with sequence coverage of approximately 140×, and using 3 ng of starting DNA.

In particular, though displaying large numbers of total mapped barcodes to the various sequence segments, only a very small percentage of overlapping barcodes, e.g., less than 0.5% of the total mapped barcoded sequences, were seen for the fusion structure by comparison to the wild type structure which demonstrated very high numbers of common or overlapping barcodes. As a result, the commonly mapping barcodes across fusion or translocation break points provides a powerful basis for identifying those translocation events.

An algorithm for SV detection was also employed that first searches for all pairs of genomic loci with significant barcode intersection/overlap, encoding this search as an efficient sparse matrix-multiplication. Candidates from this first stage are then filtered utilizing a probabilistic model that incorporates read-pair, split-read, and barcode data. SV-calling on NA12878 and NA20847, resulted in calling multiple large-scale deletions and inversions and phasing them with respect to adjacent phase blocks, showing consistency of phasing with inheritance patterns in the nuclear trio descried above.

### Example 3: Detecting Increased Susceptibility to Lupus Via CNV Screening

A patient is tested for susceptibility to lupus. Blood is drawn from the patient. A cell-free DNA sample is sequenced using techniques recited herein. The sequence is then compared to a known genome reference sequence to determine the CNV of different genes. A low copy number of FCGR3B (the CD16 cell surface immunoglobulin receptor) indicates an increased susceptibility to systemic lupus erythematosus. The patient is informed of any copy number aberrations and the associated risks/disease.

### Example 4: Detecting Increased Predisposition to Neuroblastoma Via CNV Screening

A patient is tested for predisposition to neuroblastoma. Blood is drawn from the patient. A cell-free DNA sample is sequenced using techniques recited herein. The sequence is then compared to a known genome reference sequence to determine the CNV of different genes. CNV at 1q21.1 indicates an increased predisposition to neuroblastoma. The patient is informed of any copy number aberrations and the associated risks/disease.

### Example 5: Differential Diagnosis of Lung Cancer Via CNV Screening

A patient with chromic cough, weight loss and shortness of breath is tested for lung cancer. Blood is drawn from the patient. The circulating tumor cell (CTC) or cell-free DNA sample is sequenced using techniques recited herein. The CTC sequence is then compared to a known genome reference sequence to determine the CNV of different genes. If the EGFR copy number in the DNA is higher than normal, the patient can be differentially diagnosed with non-small cell lung cancer (NSCLC) instead of small-cell lung cancer. The CTC of non-small cell lung cancer also has other copy number variations that may further distinguish it from small-cell lung cancer. Depending on the stage of the cancer, surgery, chemotherapy, or radiation therapy is prescribed.

Small cell lung cancer is most often more rapidly and widely metastatic than non-small cell lung carcinoma (and hence staged differently). NSCLCs are usually not very sensitive to chemotherapy and/or radiation, so surgery is the treatment of choice if diagnosed at an early stage, often with adjuvant (ancillary) chemotherapy involving cisplatin. Targeted therapy may also be available for patients with non-small cell lung cancer (NSCLC), for example ALK inhibitors such as Crizotinib. Targeted therapy blocks the growth of cancer cells by interfering with specific targeted molecules needed for carcinogenesis and tumor growth, rather than by simply interfering with all rapidly dividing cells (e.g. with traditional chemotherapy).

### Example 6: Differential Diagnosis of Fetal Aneuploidies Via Phasing

Fetal aneuploidies are aberrations in chromosome number. Aneuploidies commonly result in significant physical and neurological impairments. A reduction in the number of X chromosomes is responsible for Turner's syndrome. An increase in copy number of chromosome number 21 results in Down's syndrome. Invasive testing such as amniocentesis or Chorionic Villus Sampling (CVS) can lead to risk of pregnancy loss and less invasive methods of testing the maternal blood are used here.

A pregnant patient with a family history of Down's syndrome or Turner's syndrome is tested. A maternal blood sample containing fetal genetic material is collected. The nucleic acids from different chromosomes are then separated into different partitions along with barcoded tag molecules as described herein. The samples are then sequenced and the number of each chromosome copies is compared to a sequence on a normal diploid chromosome. The patient is informed of any copy number aberrations for different chromosomes and the associated risks/disease.

### Example 7: Detecting Chromosomal Translocations Via Phasing for Differential Diagnosis of Burkitt's Lymphoma

Burkitt's Lymphoma is characterized by a t(8;14) translocation in the chromosomes. A patient generally diagnosed with lymphoma is tested for Burkitt's Lymphoma. A tumor-biopsy specimen is collected from the lymph node. The nucleic acids from different chromosomes are the separated into different partitions along with barcoded tag molecules as described herein. The samples are then sequenced and compared to a control DNA sample to detect chromosomal translocation. If the patient is diagnosed as having Burkitt's Lymphoma, a more intensive chemotherapy regimen, including the CHOP or R-CHOP regimen, can be required than with other types of lymphoma. CHOP consists of. Cyclophosphamide, an alkylating agent which damages DNA by binding to it and causing the formation of cross-links; Hydroxydaunorubicin (also called doxorubicin or Adriamycin), an intercalating agent which damages DNA by inserting itself between DNA bases; Oncovin (vincristine), which prevents cells from duplicating by binding to the protein tubulin; Prednisone or prednisolone, which are corticosteroids. This regimen can also be combined with the monoclonal antibody rituximab since Burkitt's the lymphoma is of B cell origin; this combination is called R-CHOP.

### Example 8: Phasing a Fetal Genome Sequence Derived from Cell-Free DNA by Comparison to Parental Genomes

A sample comprising maternal DNA from a pregnant patient and a sample comprising paternal DNA from the father of the fetus are collected. The nucleic acids from each sample are separated into different partitions along with molecular barcoded tags as described herein. The samples are then sequenced and the sequences are used to generate inferred contigs for each of the partitioned maternal and paternal fragments. The inferred contigs are used to construct haplotype blocks for portions of each of the maternal and paternal chromosomes.

A maternal blood sample containing fetal genetic material is collected. The cell-free DNA is sequenced to generate a sequences of both the maternal circulating DNA and the fetal circulating DNA. The reads are compared to the paternal and maternal phase blocks generated above. Some phase blocks have undergone recombination during meiosis. The fetal material is identified that matches the paternal phase blocks and not the maternal phase blocks. In some cases, the fetal material matches the entirety of a paternal phase block and it is determined that the fetus has that paternal phase block in the paternally inherited chromosome. In other cases, the fetal material matches part of a phase block and then matches a second phase block, where the two phase blocks are on homologous chromosomal regions in the paternal genome. It is determined that a meiotic recombination event occurred at this region, the most likely point of recombination is determined, and a novel fetal phase block that is a combination of two paternal phase blocks is produced.

The sequences of the circulating DNA are compared to the maternal phase blocks. Sites of heterozygosity in the maternal phase blocks are used to determine the most likely phase of the maternally derived fetal chromosomes. The circulating DNA sequences are used to determine the copy number at the heterozygous sites of the maternal genome. Elevated copy numbers of specific maternal phase blocks indicates that the maternally derived chromosome of the fetus contains the sequence of the elevated phase block. In some cases, similarly to that described in the paternal case, at first one phase block of a homologous region will appear elevated, and then a portion of another phase block of the same region will appear elevated, indicating that meiotic recombination has occurred. In these cases, a the most likely region of recombination is determined and a new fetal phase block is constructed from the two maternal phase blocks.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A method of nucleic acid analysis, comprising:
(a) providing:
(i) a plurality of fragment molecules deriving from different nucleic acid molecules, wherein given fragment molecules of the plurality of fragment molecules are derived from a given nucleic acid molecule of the different nucleic acid molecules;
(ii) a plurality of oligonucleotides, wherein each oligonucleotide of the plurality of oligonucleotides comprises a barcode sequence and a random primer sequence, wherein the barcode sequence and the random primer sequence are on a same molecule; and
(b) generating a plurality of barcoded fragment molecules using the plurality of fragment molecules and the plurality of oligonucleotides by (i) hybridizing random primer sequences of the plurality of oligonucleotides to multiple different locations of the plurality of fragment molecules and (ii) performing nucleic acid extension reactions using the random primer sequences and using the plurality of fragment molecules as templates;
wherein barcoded fragment molecules generated from the given fragment molecules comprise a common barcode sequence that identifies the barcoded fragment molecules as derived from the given nucleic acid molecule.

2. The method of claim 1, further comprising (c) sequencing the plurality of barcoded fragment molecules or derivatives thereof to provide a plurality of fragment sequences, wherein fragment sequences of the plurality of fragment sequences that correspond to the given fragment molecules each comprises the common barcode sequence or a reverse complement thereof.

3. The method of claim 2, further comprising (d) linking the fragment sequences comprising the common barcode sequence or the reverse complement thereof in one or more inferred contigs.

4. The method of claim 2, wherein the given nucleic acid molecule comprises a structural variation, and wherein the method further comprises analyzing the plurality of fragment sequences and determining a presence of the structural variation based on an amount of the fragment sequences that comprise the common barcode sequence or the reverse complement thereof.

5. The method of claim 4, wherein the structural variation comprises a copy number variation, an insertion, a deletion, a translocation, an inversion, a rearrangement, a repeat expansion, a duplication, retrotransposon, or a gene fusion.

6. The method of claim 4, wherein the structural variation is associated with a disease or a disorder.

7. The method of claim 2, wherein the given nucleic acid molecule is a chromosome.

8. The method of claim 7, further comprising analyzing the plurality of fragment sequences and determining that the fragment sequences that comprise the common barcode sequence or reverse complement thereof are derived from the chromosome.

9. The method of claim 1, wherein other fragment molecules of the plurality of fragment molecules, separate from the given fragment molecules, are derived from another nucleic acid molecule of the different nucleic acid molecules, and wherein other barcoded fragment molecules generated from the other fragment molecules comprise a different barcode sequence.

10. The method of claim 9, wherein the given nucleic acid molecule comprises a structural variation and the other nucleic acid molecule does not comprise the structural variation.

11. The method of claim 9, wherein the given nucleic acid molecule is a first chromosome and the other nucleic acid molecule is a second chromosome.

12. The method of claim 9, wherein the given nucleic acid molecule is a fetal nucleic acid molecule from a fetus and the different nucleic acid molecule is a maternal nucleic acid molecule from a pregnant mother of the fetus.

13. The method of claim 1, wherein the plurality of fragment molecules and the plurality of oligonucleotides in (a) are provided in multiple partitions, wherein the given fragment molecules of the plurality of fragment molecules are co-partitioned with oligonucleotides comprising the common barcode sequence or a reverse complement thereof in a given partition of the multiple partitions.

14. The method of claim 13, wherein each partition of the multiple partitions comprises oligonucleotides comprising a barcode sequence that is different from barcode sequences in other partitions of the multiple partitions.

15. The method of claim 13, wherein the multiple partitions are droplets or wells.

16. The method of claim 1, further comprising fragmenting the different nucleic acid molecules to generate the plurality of fragment molecules.

17. The method of claim 16, further comprising partitioning the plurality of fragment molecules into multiple partitions.

18. The method of claim 1, wherein the plurality of oligonucleotides are releasably attached to beads.

19. The method of claim 1, wherein the given nucleic acid molecule is derived from cell-free nucleic acid.

20. The method of claim 1, wherein the given nucleic acid molecule is a tumor nucleic acid molecule.
